(19) United States
(12) Patent Application Publication
(10) Pub. No.: US 2025/0057466 A1
(43) Pub. Date: Feb. 20, 2025

(54) SYSTEM FOR DIAGNOSING, TRACKING, AND PREDICTING RECOVERY PATTERNS IN PATIENTS WITH TRAUMATIC BRAIN INJURY

(71) Applicants: Arizona Board of Regents on behalf of Arizona State University, Scottsdale, AZ (US); Mayo Foundation for Medical Education and Research, Rochester, MN (US)

(72) Inventors: Visar BERISHA, Tempe, AZ (US); Jianwei ZHANG, Phoenix, AZ (US); Todd J. SCHWEDT, Scottsdale, AZ (US); Catherine CHONG, Cave Creek, AZ (US); Suren JAYASURIYA, Tempe, AZ (US); Teresa Wu, Gilbert, AZ (US)

(21) Appl. No.: 18/821,111

(22) Filed: Aug. 30, 2024

Related U.S. Application Data
(63) Continuation of application No. PCT/US2023/063497, filed on Mar. 1, 2023.
(60) Provisional application No. 63/315,977, filed on Mar. 2, 2022.

(51) Int. Cl. A61B 5/00; G06N 20/00

(52) U.S. Cl. CPC A61B 5/4064; A61B 5/4803; G06N 20/00

(57) ABSTRACT

Described are platforms, systems, media, and methods for evaluating, monitoring, and/or treating a subject for brain injury based on machine learning analysis of one or more of brain imaging features, clinical features, demographic features, or speech features.

### CROSS-REFERENCE

This application is a continuation of International Patent Application No. PCT/US2023/063497, filed Mar. 1, 2023, which claims the benefit of U.S. Provisional Application No. 63/315,997 filed Mar. 2, 2022, which is incorporated herein by reference in its entirety.

This invention was made with government support under NS113315 awarded by National Institutes of Health. The US government has certain rights in the invention.

### BACKGROUND

Individuals with migraine report changes in speech during migraine attacks and several studies have documented speech difficulty during the aura phase of the attack as well as prior to and during the attack. Although severe alterations in speech are easily observed, objective methods for assessing subtle changes in speech production in subjects with headache remain sparse. Post-traumatic headache (PTH) due to mild traumatic brain injury (mTBI) commonly has symptoms that are similar to those of migraine. In fact, most subjects with PTH have migraine-like headache features.

### SUMMARY

In some aspects, the platforms, systems, and methods disclosed herein enable classification of individuals with PTH based on alterations in brain structure and/or function, and clinical features such as headache, psychological, cognitive, and speech features compared to healthy controls without history of mTBI. In some embodiments, to determine speech changes in individuals with PTH, a speech elicitation task embedded within a mobile app was used to assess objective measures of speech that relate to a combination of motor and cognitive-linguistic components of speech. These include sentence speaking rate, pause rate during spontaneous speech production, pitch (average pitch and pitch variance), vowel and consonant articulation precision, and vowel space area. These measures were collected to: 1) investigate speech differences between individuals with PTH and healthy controls, and 2) assess whether individuals with PTH have speech changes during headaches compared to when they are headache-free. In some embodiments, brain imaging data is collected and processed using one or more image-based machine learning models to determine an indication associated with brain injury based on one or more brain structure and/or function features relevant to brain injury. The brain imaging data can be, for example, MR imaging. In some embodiments, clinical data is collected and processed using one or more machine learning models to determine an indication associated with brain injury based on clinical features such as headache, psychological, cognitive, and/or speech features.

Objective features measured from data such as brain imaging data, clinical data such as doctor diagnostic data or speech samples obtained from individuals with acute PTH can be used to provide a surrogate measure of headache burden, which could have utility in the future for tracking headache persistence and recovery.

Disclosed herein, in one aspect is, a method for evaluating a subject for brain injury, comprising: (a) receiving input data comprising one or more acoustic features and/or one or more linguistic features extracted from audio data obtained for said subject; (b) processing the input data using a machine learning module configured to evaluate acoustic and/or linguistic features; (c) generate an evaluation of said subject based on said processing of said input data using said machine learning module, said evaluation comprising an indication of brain injury. In some embodiments, further comprising pre-determining said subject has suffered an injury affecting brain health and selecting said subject for said evaluation. In some embodiments, said injury comprises traumatic brain injury or mild traumatic brain injury. In some embodiments, further comprising prompting said subject for said audio data. In some embodiments, said subject is shown one or more sentences and prompted to read said one or more sentences. In some embodiments, further comprising capturing said audio data using a microphone, said audio data comprising an audio recording of said subject reading said one or more sentences. In some embodiments, further comprising processing said audio data to extract said one or more acoustic features and/or one or more linguistic features. In some embodiments, said one or more acoustic features comprises one or more of sentence speaking rate, average pitch value, pitch variance, vowel and/or consonant articulation precision, vowel space area, or spontaneous pause rate. In some embodiments, said one or more linguistic features comprises one or more of LIWC feature, part of speech feature, language complexity feature, grammatical constituent feature, or phrase formation type. In some embodiments, said LIWC feature comprises a calculation of words categorized according to one or more of attention and/or focus, emotion, social relationship, thinking style, or cognitive complexity. In some embodiments, said part of speech feature comprises a percentile of total words for a part of speech. In some embodiments, said language complexity feature comprises a Yngve Depth, Brunet Index, or Honore Statistic. In some embodiments, said part of speech feature comprises an appearance time of a noun phrase, an appearance time of a verb phrase, or an appearance time of a noun phrase that contains only one noun. In some embodiments, said machine learning module comprises a model trained using a training data set comprising acoustic and/or linguistic features for a plurality of individuals, wherein each of said plurality of individuals is labeled according to said indication of brain injury. In some embodiments, said indication of brain injury is associated with traumatic brain injury. In some embodiments, said indication of brain injury comprises one or more symptoms of traumatic brain injury. In some embodiments, said indication of brain injury is associated with a severity of traumatic brain injury. In some embodiments, said indication of brain injury comprises a prediction of a future symptom of traumatic brain injury. In some embodiments, said indication of brain injury differentiates between whether said subject will recover from an acute traumatic brain injury or whether said subject will suffer one or more symptoms of said acute traumatic brain injury. In some embodiments, said one or more symptoms of acute traumatic brain injury comprises post-traumatic headache or persistent post-traumatic headache. In some embodiments, further comprising monitoring said subject over time through repeated or periodic collection and processing of additional input data. In some embodiments, further comprising collecting clinical information or subject feedback on one or more symptoms of brain injury for said subject. In some embodiments, further comprising collecting physician diagnostic data for said subject. In some embodiments, further comprising generating an indication of recovery progress based on said audio data and said additional audio data. In some embodiments, further comprising recommending treatment for said brain injury based on said evaluation. In some embodiments, further comprising providing treatment for said brain injury based on said evaluation. In some embodiments, said subject is treated based on said evaluation, wherein said method further comprises monitoring said subject during or after treatment through collection and processing of additional input data to determine treatment efficacy. Disclosed herein, in another aspect, is a computer-implemented system comprising: a digital processing device comprising: at least one processor, an operating system configured to perform executable instructions, a memory, and a computer program including instructions executable by the digital processing device to create an application configured to perform any of the methods for evaluating a subject for brain injury disclosed herein. Disclosed herein, in another aspect, is a non-transitory computer-readable storage media encoded with a computer program including instructions executable by a processor to create an application configured to perform any of the methods for evaluating a subject for brain injury disclosed herein.

Disclosed herein, in another aspect, is a method for evaluating a subject for brain injury, comprising: (a) receiving input data for said subject comprising a panel of biomarkers comprising one or more brain imaging features, clinical features, demographic features, or speech features; (b) processing the input data using a machine learning module configured to evaluate said panel of biomarkers; (c) generate an evaluation of said subject based on said processing of said input data using said machine learning module, said evaluation comprising an indication of brain injury. In some embodiments, further comprising pre-determining said subject has suffered an injury affecting brain health and selecting said subject for said evaluation. In some embodiments, said injury comprises traumatic brain injury or mild traumatic brain injury. In some embodiments, further comprising collecting brain imaging data for said subject. In some embodiments, said brain imaging data comprises magnetic resonance imaging (MRI) image(s). In some embodiments, said brain imaging data is collected between 7 and 28 days after onset of post-traumatic headache in said subject. In some embodiments, said one or more brain imaging features comprises one or more brain structure features and/or brain function features. In some embodiments, said brain structure features comprises gray matter volume, gray matter area, gray matter thickness, white matter tract integrity, or any combination thereof. In some embodiments, said brain function features comprises functional connectivity, brain perfusion, or any combination thereof. In some embodiments, further comprising collecting clinical data for said subject. In some embodiments, one or more said clinical features comprises clinical data gathered using a headache symptom battery test. In some embodiments, further comprising prompting said subject for audio data comprising said speech features. In some embodiments, said subject is shown one or more sentences and prompted to read said one or more sentences. In some embodiments, further comprising capturing said audio data using a microphone, said audio data comprising an audio recording of said subject reading said one or more sentences. In some embodiments, further comprising processing said audio data to extract said one or more acoustic features and/or one or more linguistic features. In some embodiments, said one or more speech features comprises articulation entropy, speaking rate, pause rate, articulation rate, vowel space area, energy decay slope, phonatory duration, or average pitch. In some embodiments, said machine learning module comprises a model generated using elastic net regression or decision tree machine learning algorithm. In some embodiments, said one or more speech features comprises at least one acoustic feature comprising one or more of sentence speaking rate, average pitch value, pitch variance, vowel and/or consonant articulation precision, vowel space area, or spontaneous pause rate. In some embodiments, said one or more speech features comprises at least one linguistic feature comprising one or more of LIWC feature, part of speech feature, language complexity feature, grammatical constituent feature, or phrase formation type. In some embodiments, said LIWC feature comprises a calculation of words categorized according to one or more of attention and/or focus, emotion, social relationship, thinking style, or cognitive complexity. In some embodiments, said part of speech feature comprises a percentile of total words for a part of speech. In some embodiments, said language complexity feature comprises a Yngve Depth, Brunet Index, or Honore Statistic. In some embodiments, said part of speech feature comprises an appearance time of a noun phrase, an appearance time of a verb phrase, or an appearance time of a noun phrase that contains only one noun. In some embodiments, said machine learning module comprises one or more models trained using a training data set comprising data corresponding to said panel of biomarkers for a plurality of subjects, wherein each of said plurality of individuals is labeled according to said indication of brain injury. In some embodiments, at least one of said one or more models is configured to process a reduced feature set identified through principal component analysis (PCA). In some embodiments, at least one of said one or more models is generated using a classification algorithm. In some embodiments, said classification algorithm comprises linear discriminant analysis, quadratic discriminant analysis, or support vector machine. In some embodiments, said classification algorithm is performance optimized using a particle swarm optimization (PSO) technique. In some embodiments, said biomarker panel is an ensemble biomarker panel comprising two or more of brain imaging features, clinical features, demographic features, or speech features. In some embodiments, said machine learning module comprises an ensemble learning technique combining predictions, wherein said each prediction is generated by a classifier for a feature category of said ensemble biomarker panel. In some embodiments, said indication of brain injury is associated with traumatic brain injury. In some embodiments, said indication of brain injury comprises one or more symptoms of traumatic brain injury. In some embodiments, said indication of brain injury is associated with a severity of traumatic brain injury. In some embodiments, said indication of brain injury comprises a prediction of a future symptom of traumatic brain injury. In some embodiments, said machine learning module comprises an ensemble modeling technique using a plurality of trained models. In some embodiments, said plurality of trained models comprises a first model configured to evaluate said brain imaging features and a second model configured to evaluate said speech features In some embodiments, said indication of brain injury differentiates between whether said subject will recover from an acute traumatic brain injury or whether said subject will suffer one or more symptoms of said acute traumatic brain injury. In some embodiments, said one or more symptoms of acute traumatic brain injury comprises post-traumatic headache or persistent post-traumatic headache. In some embodiments, further comprising monitoring said subject over time through repeated or periodic collection and processing of additional input data. In some embodiments, further comprising collecting clinical information or subject feedback on one or more symptoms of brain injury for said subject. In some embodiments, further comprising collecting physician diagnostic data for said subject. In some embodiments, further comprising generating an indication of recovery progress based on said audio data and said additional audio data. In some embodiments, further comprising recommending treatment for said brain injury based on said evaluation. In some embodiments, further comprising providing treatment for said brain injury based on said evaluation. In some embodiments, said subject is treated based on said evaluation, wherein said method further comprises monitoring said subject during or after treatment through collection and processing of additional input data to determine treatment efficacy. Disclosed herein, in another aspect, is a computer-implemented system comprising: a digital processing device comprising: at least one processor, an operating system configured to perform executable instructions, a memory, and a computer program including instructions executable by the digital processing device to create an application configured to perform any of the methods for evaluating a subject for brain injury disclosed herein. Disclosed herein, in another aspect, is a non-transitory computer-readable storage media encoded with a computer program including instructions executable by a processor to create an application configured to perform any of the methods for evaluating a subject for brain injury disclosed herein.

### BRIEF DESCRIPTION OF THE DRAWINGS

The novel features of the disclosure are set forth with particularity in the appended claims. The file of this patent contains at least one drawing/photograph executed in color. Copies of this patent with color drawing(s)/photograph(s) will be provided by the Office upon request and payment of the necessary fee. A better understanding of the features and advantages of the present disclosure will be obtained by reference to the following detailed description that sets forth illustrative embodiments, in which the principles of the disclosure are utilized, and the accompanying drawings of which:

FIG. 1 shows a methodology for extracting and normalizing speech features, according to some embodiments.

FIG. 2 shows spontaneous pause rate as measured from a spontaneous speaking task, according to some embodiments.

FIG. 3 shows sentence speaking rate, vowel articulation precision, and consonant articulation precision measurements from sample audio data, according to some embodiments.

FIG. 4 shows data collected from individuals with concussion and persistent PTH that demonstrate regions of less cortical thickness (shown by the color ‘red’ on FIG. 4) in patients with PPTH relative to healthy controls, according to some embodiments.

FIG. 5 shows a DTI analysis for Fibertract alterations in patients with Acute PTH, according to some embodiments.

FIG. 6 shows DTI analysis for Fibertract alterations in patients with PPTH, according to some embodiments.

FIG. 7 illustrates brain regions that showed differences in brain structure between migraine and PPTH, including the right lateral orbitofrontal lobe, left caudal middle frontal lobe, left superior frontal lobe, left precuneus and right supramarginal gyrus, according to some embodiments.

FIG. 8 shows regions of the brain relevant for distinguishing chronic migraine patients from healthy controls based on structural data, according to some embodiments.

FIG. 9 shows a comparison of the cohorts with headache, psychological, cognitive, and headache/psychological/cognitive symptoms.

FIG. 10 shows a non-limiting example of a computing device; in this case, a device with one or more processors, memory, storage, and a network interface, per one or more embodiments herein;

FIG. 11 shows a non-limiting example of a web/mobile application provision system; in this case, a system providing browser-based and/or native mobile user interfaces, per one or more embodiments herein; and

FIG. 12 shows a non-limiting example of a cloud-based web/mobile application provision system; in this case, a system comprising an elastically load balanced, auto-scaling web server and application server resources as well synchronously replicated databases, per one or more embodiments herein.

### DETAILED DESCRIPTION

Disclosed herein are platforms, systems, and methods for evaluating a subject for brain or cognitive health associated with trauma using a biomarker panel comprising one or more categories of features. The biomarker panel can include brain imaging features, clinical features, and/or speech features, one or more of which may be processed and analyzed using an ensemble machine learning technique to produce accurate evaluations of brain or cognitive health. The biomarker panel can include speech patterns or signatures based on linguistic and/or acoustic features obtained from speech samples of the subject. In some embodiments, provided herein is a speech assessment tool to evaluate brain or cognitive health associated with trauma. The evaluation can include identification of a subtype of brain health associated with future cognitive effects. In some cases, the speech assessment tool is configured to determine if patients with mild traumatic brain injury (mTBI) and a subsequent post-traumatic headache (PTH) have distinctive speech changes. Determining differences in mTBI patients who have PTH and those who do not, and having a method for predicting PTH persistence vs. resolution, can help to aid management of mTBI patients. Currently there is no technology available to differentiate between the patients who would recover from TBI and those who would develop post traumatic symptoms like headache following TBI (e.g., acute and/or persistent post traumatic headaches). The platforms, systems, and methods disclosed herein enable features such as acoustic and linguistic features abstracted from speech samples obtained from patients to be used as biomarkers for clinical trials assisting diagnosis and tracking recovery from TBI. Other applications include detection, evaluation, or diagnosis of patients for a traumatic brain injury as well as clinical management of patients, for example, monitoring patient health status over time. Neuroimaging data can also be used in conjunction with the speech therapy tool to determine a unique biomarker signature of mTBI patients with PTH.

In some embodiments, brain imaging data is used to generate one or more brain structure and/or function features included in the biomarker panel. The brain imaging data can be acquired using magnetic resonance imaging (MRI).

In some embodiments, clinical data is used to generate one or more clinical features included in the biomarker panel. Clinical data can be collected using tests such as a symptom battery or doctor's diagnostic information (e.g., electronic health record from a doctor's visit diagnosing or evaluating the subject following TBI).

In some embodiments, the platform, system, or method comprises recording headache status followed by speech sample collection using PTH Speech app every 3 days within 90 days after meeting the doctor, both of which are used for speech feature abstraction.

In some embodiments, when spontaneous speech content is related to headache, 2 files of spontaneous speech files for each submission are transcribed via automatic speech recognition or secured artificial transcription.

In some embodiments, text features such as emotional characteristics, phrase formation, and other relevant features are abstracted using Stanford Natural Language process (NLP) from spontaneous speech transcription.

In some embodiments, the acoustic features comprise at least 1, 2, 3, 4, 5, or 6 features. Examples of acoustic features include speak rate, pause rate, and average pitch value, which can be abstracted from one or more sentence reading speech files and/or one or more spontaneous speech files from each patient. As an example, acoustic features may be extracted from 5 sentence reading speech files and 2 spontaneous speech files.

Using a linguistic and acoustic feature extraction algorithm, patients with post traumatic headache (PTH) were determined to have had significantly less precision for vowel and consonant articulation and longer spontaneous pause rates than healthy controls.

The use of speech biomarkers as provided herein enables clinicians to build a diagnosis model, track recovery from TBI, and/or deliver targeted treatment to patients who are likely to develop persistent post traumatic symptoms.

### Acoustic Features Extraction

The platforms, systems, and methods disclosed herein can utilize one or more acoustic extraction algorithms to generate one or more acoustic features suitable for use as input data for a trained algorithm or model. Non-limiting examples of acoustic features include sentence speaking rate, average pitch, pitch variance, vowel and/or consonant articulation precision, vowel space area, and spontaneous pause rate. In some cases, normalization is performed on acoustic features.

In some embodiments, sentence speaking rate for one sentence speaking record is calculated as the number of syllables (e.g., determined from a sentence reading prompt) divided by the speaking time. Speaking time can be determined by a voice activity detection algorithm.

In some embodiments, average pitch and/or pitch variance is extracted from one or more speaking records. As a non-limiting example, a pitch estimator such as Google's REAPER can be used to extract the pitch contour from the speaking records. Average pitch and pitch variance can be calculated from the extracted pitch contour.

In some embodiments, vowel and/or consonant articulation precision can be generated (e.g., as a score) using the audio files (e.g., sentence reading record(s)) and corresponding texts (e.g., sentence texts). As a non-limiting example, vowel and/or consonant articulation precision can be estimated using the goodness of pronunciation (GOP) score evaluation algorithm 16 to generate vowel and consonant articulation precision scores.

In some embodiments, vowel space area can be extracted from an audio file (e.g., sentence reading record) using a vowel space extraction algorithm.

In some embodiments, spontaneous pause rate is calculated using a voice activity detection algorithm which detects the timepoints of participant start or stop speaking in spontaneous speech records. The pause time is identified as the non-voice period during spontaneous speech. The spontaneous pause rate can be calculated as the ratio of pause time over total speaking time.

In some embodiments, normalization is performed on one or more acoustic features such as, for example, sentence speaking rate, average pitch and/or pitch variance, and vowel and/or consonant articulation precision. One or more acoustic features can be normalized by demographic parameters such as the subject's age and sex. An estimate of the cumulative distribution function (CDF) can be computed from the subset of individuals in a database of speech data that are matched based on the demographic parameters or other relevant parameters. The features can then be converted to percentiles relative to the CDF, and the normalized percentiles can then be used as features. As a non-limiting example, the Mozilla common voice English database provides a large open-source corpus for speech data that enables normalization according to various parameters. The nonparametric estimate of the CDF was computed from a subset of age/sex matched individuals in Mozilla. The features were then converted to percentiles relative to this CDF and the normalized percentiles were then used as features.

### Linguistic Features Extraction

The platforms, systems, and methods disclosed herein can utilize a speech transcription algorithm to generate a transcript of recorded audio data. In some cases, the audio data comprises spontaneous speech that is not deliberately elicited from a user or speaker. For example, a smartphone may have installed a mobile app that is set to automatically record audio of spontaneous speech that is detected by its microphone and/or automatically transcribe spontaneous speech. In some cases, audio data can be outsourced for transcription. One example of an available transcription service is the GoTranscript service.

In some embodiments, one or more linguistic features suitable as input data for a trained model are extracted from the speech transcript(s). A program or algorithm can be used to extract one or more linguistic features. Examples of linguistic features include LIWC features, part of speech features, language complexity features, and grammatical constituent features. In some cases, a text analysis program or algorithm such as Linguistic Inquiry and Word Count (LIWC) is used to counts words in various categories that are meaningful. Examples of such categories include attention and/or focus, emotion (e.g., positive or negative emotional words), social relationships, thinking styles, cognitive complexity, and other relevant categories. As a non-limiting example, the Linguistic Inquiry and Word Count (LIWC2015) program can be used to extract or calculate the number of different emotional words from spontaneous speech transcripts.

In some embodiments, one or more part of speech features are used as input data for a trained model. In some cases, words in a transcript are assigned a corresponding part of speech (e.g., noun, verb, adjective, adverb, etc.). The percentile for each part of speech (e.g., verb) out of all words may be calculated and used for the analysis. In some cases, the percentile for a given part of speech is a feature used as input data for an algorithm or model. As a non-limiting example, the Stanford Log-linear Part-Of-Speech Tagger can be used for reading transcripts and assigning parts of speech to each word.

In some embodiments, one or more language complexity features are used as input data for a trained model. As a non-limiting example, the Stanford Parser can be used for determining the grammatical structure of spontaneous speech transcripts. The generated parsing tree, i.e., the results of grammatical structure, can be used for calculating the natural language complexity features. Non-limiting examples of language complexity features include Yngve Depth, Brunet Index, Honore Statistics, etc.

In some embodiments, one or more grammatical constituent features are used as input data for a trained model. The generated parsing tree can be used to calculate the appearance times of certain grammar structures. For example, the appearance times of noun phrase, verb phrase, the noun phrase that only contains one noun, and other grammatical structures can be relevant grammatical constituent features.

### Diagnosis of Post-Traumatic Symptoms

In some aspects, disclosed herein are platforms, systems, and methods for evaluating the brain injury of a subject based on audio data. The brain injury can be a post-traumatic brain injury such as mild post-traumatic brain injury. The severity of the post-traumatic brain injury may correlate with symptoms such as post-traumatic headache. Although a diagnosis of post-traumatic headache can be clinically straightforward in a patient who has never suffered headaches prior to a concussion, the diagnosis of post-traumatic headache is more difficult to make in a patient who has had an existing history of migraine prior to suffering a concussion. Additionally, the diagnosis of post-traumatic symptoms including headache relies on patient history, which can be biased or inaccurate. As there are currently no objective biomarkers for confirming or diagnosing post-traumatic headache, disclosed herein is a diagnostic or evaluation process based on speech patterns would solve this dilemma and could be used as an objective test in the clinical setting and/or as a side-line field test for the assessment of concussion-related symptoms such as persistent post-traumatic brain injury headache.

Input data including various acoustic and/or linguistic speech features, a label or classification of the brain injury (e.g., headache status), optionally including correlated doctors' diagnosis information and/or clinical indicators for post-traumatic symptoms diagnosis, can be evaluated to identify the features most related to the diagnosis results. These features can make up a panel of biomarkers used by a trained machine learning model for evaluation of brain injury such as a post-traumatic symptom diagnosis. The machine learning module comprising a trained diagnostic model can automatically process the panel of biomarkers to generate an evaluation comprising an indication of brain injury (e.g., presence of brain injury, prediction of future symptoms such as headache, determination of progress or recovery, etc.). During implementation, the platforms, systems, or methods disclosed herein can prompt the diagnostic results for doctors to refer to after a subject/patient submits speech or audio data (e.g., repeatedly or periodically for a period of time).

Tracking Recovery from Brain Injury (TBI)

In some aspects, the platforms, systems, and methods disclosed herein provide monitoring and/or tracking of recovery from brain injury through audio-based evaluation. Longitudinal analysis can be performed on various acoustic and/or linguistic features together with data collected on the brain injury such as headache intensity and TBI-related symptom severity and/or doctors' diagnostic information in order to identify the speech features pattern most correlated with recovery. The pattern of features can then be identified through longitudinal monitoring of the subject to determine if the subject has a consistent speech features pattern with the identified speech features pattern after speech data for a period of time has been obtained. The output can then automatically generate an indication that there is a recovery trend for the patient.

Prediction of Recovery from Brain Injury (TBI)

In some aspects, disclosed herein are platforms, systems, and methods for predicting recovery from brain injury based on audio data. It is often impossible for the clinician to provide the subject/patient with accurate information relative to his/her recovery process. Not knowing whether a subject is going to recover on their own within days, weeks, or months or will develop persistent post-traumatic symptoms prevents the clinician to administer early treatment to those subjects who would benefit most.

The identification of one or more acoustic and/or linguistic features that serve as speech biomarkers for developing persistent post-traumatic symptoms including headache would enable the physician to deliver targeted treatments to patients at high-risk for developing persistent post-traumatic symptoms including headache and to avoid treating patients who would quickly recover on their own. A machine learning model can be trained to process audio data and generate predictions or evaluations relating to brain injury. The machine learning model can be a memory-enabled machine learning model, for example, a long short term memory (LSTM) model, based on the longitudinal speech features. An LSTM model is an artificial recurrent neural network architecture used in deep learning with feedback connections capable of processing sequences of data such as speech data. The model can be trained to predict the average headache frequency, intensity or symptom severity in a few days or weeks based on the longitudinal speech features over a period of time. These prediction results can be further used to predict whether the subject can recover within a period of time or will develop persistent post-traumatic symptoms. During implementation, the validated memory-enable machine learning model can analyze speech features such as one or more acoustic and/or linguistic features and provide the prediction results to the subject and/or their doctor or healthcare provider for their reference to inform their medical decision-making and advice.

Altered Speech Patterns in Subjects with Post-Traumatic Headache Due to Mild Traumatic Brain Injury

### Methods

This study received approval from the Mayo Clinic IRB in 2019. All subjects completed written informed consent. Subjects had to be native English-speakers aged 18-65 years. All subjects were required to have a mobile device with capability for downloading an application used to collect speech and had to be willing and able to provide a speech sample once every 3 days over a period of 3 months. Subjects with PTH were eligible for enrollment starting on the day of mTBI and until 59 days post-mTBI. Subjects had to meet criteria for acute PTH attributable to mTBI in accordance with the ICHD-3 criteria. For individuals with PTH, history of headache or migraine was allowed. Healthy controls had to have no history of TBI and no history of migraine. Exclusion criteria for subjects with PTH and healthy controls included the following: history of severe psychiatric disorder or neurological disorder (other than mTBI and headaches in the PTH group) and history of a speech or language disorder. Study questionnaires: Subjects with PTH completed a detailed headache symptom questionnaire. All subjects completed the Ohio State University TBI Identification Method, a standardized questionnaire assessing the lifetime history of TBI for an individual (available at www.brainline.org), the Symptom Evaluation (step 2) of the Sports Concussion Assessment Tool (SCAT-5) questionnaire, the Beck Depression Inventory (BDI) for assessing levels of depression, and the Delayed Auditory Verbal Learning Test (RAVLT, delayed recall, z-scored) for assessing verbal learning and memory. The Symptom Evaluation of the SCAT-5 is a 22 item self-report of TBI symptoms. Participants rate each item based on how they feel on a 7-point Likert scale (0 (none) to 6 (severe)). Two totals are counted: total number of symptoms (0-22) and symptom severity (0-132). The BDI is a 21-item self-report to assess symptoms of depression. Each item is scored on a value of 0 to 3 and scores are combined for a total score (0-63). Scores between 0 and 13 identify no depression, 14-19 mild depression, 20-28 moderate depression and 29-63 severe depression.

For the RAVLT, a list of 15 words is read out loud by the examiner and the examinee is asked immediately afterward to recall as many words as they can. The list is read five times and each time, the examinee is asked to recall as many words from the list as they can, in any order. Next, a distractor list is read out loud, and the participant is asked to recall only the words from the distractor list. Afterward, the participant is then asked to recall only those words from the first list, which was read 5 times. After a delay of about 20 min (delayed recall), the examinee is asked again to recall as many words as possible from the first list. Only the delayed recall z-scores, which are a measure of episodic memory performance were included in this study.

At the first study visit, subjects were taught to download the speech application to their mobile devices. The study coordinator modeled the completion of speech elicitation tasks and the correct procedure for using the speech app. This included, selecting a time and place that is comfortable, without distractions and with minimal background noise. All subjects were asked to submit a speech sample every 3 days, beginning on the day of the first study visit and continuing over a period of the subsequent 12 weeks. As it was assumed that subjects with PTH would show the most significant speech changes during the acute phase of mTBI, therefore only the speech samples submitted during the first 30 days were used for comparison between subjects with PTH and healthy controls. When comparing subjects with PTH during headache to the headache-free phase, speech samples submitted over the first 90 days were used to increase the number of available samples.

### Speech Assessment

The speech application was specifically developed for the objective evaluation of the following measures: sentence speaking rate, average pitch, pitch variance, vowel space area vowel and consonant articulation precision, and the spontaneous pause rate. As part of the speech app, subjects were asked to read out loud five sentences (sentence reading task) and to use spontaneous speech to describe activities of the previous day (spontaneous speaking task). The entire speech elicitation task took approximately 3 min to complete. Prior to starting the speech task, all subjects indicated whether they currently had a headache or whether they were headache free. If a current headache was reported then individuals were prompted to rate their headache intensity on a scale ranging from 1 (mild headache) to 10 (most severe headache imaginable). Table 1 shows a description of the speech measures that were extracted from the speech application.

TABLE 1
Description of speech features that were extracted from the speech application.

| | |
|---|---|
| Spontaneous | The ratio of pause time over total speaking time for the |
| Pause Rate | spontaneous speaking task “Recall what you did yesterday”. |
| (Rate and Rhythm) | Unit of measurement = unitless (ratio of two durations) |
| Sentence | A speaker's sentence reading speed. Higher values indicate |
| Speaking Rate | faster speaking rates. Measured from sentence reading tasks. |
| (Rate and Rhythm) | Unit measurement = syllables/second |
| Average Pitch | Pitch is the rate at which the vocal folds vibrate. Higher pitch |
| (Pitch and | values indicate a higher tone (e.g., women have a higher pitch |
| Intonation) | than men). Measured from sentence reading tasks. |
| | Unit of measurement = Hz |
| Pitch Variance | The pitch variance is a measure of prosodic variation. It |
| (Pitch and | assesses how animated a person is as they speak. A person with |
| Intonation) | flat intonation has a low pitch variance. This is measured from |
| | the sentence reading tasks. |
| | Unit of measurement = Hz² |
| Vowel Space | Two-dimensional area bounded by lines connecting first and |
| Area (VSA) | second formant frequency coordinates (F1/F2) of vowels. In |
| (Precision of | general, larger VSA in speech results in clearer and more |
| Articulation) | intelligible speech patterns compared to speech associated with |
| | smaller VSAs. Measured from sentence reading tasks. |
| | Unit of measurement = Hz² |
| Vowel | This measure gives the likelihood that the phonemes produced |
| Articulation | by a speaker sound like the phoneme that should have been |
| Precision | spoken. It is a measure of how precisely an individual |
| (Precision of | pronounces individual phonemes. Vowel articulation precision |
| Articulation) | is the average pronunciation score across all spoken vowels. It |
| | is measured with goodness of pronunciation (GOP) score. This |
| | is measured from the sentence reading task. |
| | Unit of measurement = unitless (likelihood ratio) |
| Consonant | This measure gives the likelihood that the phonemes produced |
| Articulation | by a speaker sound like the phoneme that should have been |
| Precision | spoken. It is a measure of how precisely an individual |
| (Precision of | pronounces individual phonemes. Consonant articulation |
| Articulation) | precision is the average pronunciation score across all spoken |
| | vowels. It is measured with goodness of pronunciation (GOP) |
| | score. This is measured from the sentence reading task. |
| | Unit of measurement = unitless (likelihood ratio) |

The calculations for the sentence speaking rate, average pitch, pitch variance, vowel space area, vowel and consonant articulation precision were extracted from reading the following five sentences.
1. The supermarket chain shut down because of poor management.
2. Much more money must be donated to make this department succeed.
3. In this famous coffee shop, they serve the best donuts in town.
4. The chairman decided to pave over the shopping center garden.
5. The standards committee met this afternoon in an open meeting.

### Speech Features Extraction and Normalization

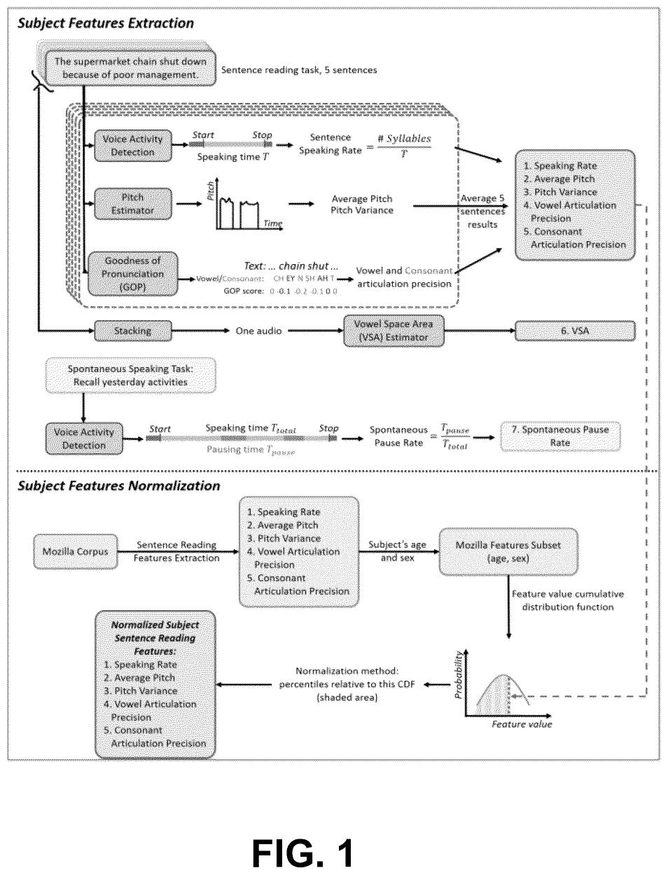

The methodology for extracting and normalizing speech features is shown in FIG. 1. After downloading the data from the server, the sentence reading audio samples (5 files) and spontaneous speaking audio sample (1 file) were used for speech feature extraction. The sentence speaking rate, average pitch, pitch variance, vowel and consonant articulation precision (measured with goodness of pronunciation scores) and vowel space area were extracted from the sentence reading tasks. The spontaneous pause rate was extracted from the spontaneous speaking task. The implementation details are described in the next section.

### Sentence Speaking Rate

First, the total speaking time in a sentence audio sample was detected by using a Voice Activity Detection (VAD) algorithm that identified the start and stop points of speech. The speaking rate was then calculated as the number of syllables (determined from the sentence reading prompt) divided by the speaking time. Consider the example of an individual reading the sentence “the supermarket chain shut down because of poor management” for 4.01 s. As there are a total of 15 syllables in the sentence: “the su-per-mar-ket chain shut down be-cause of poor man-age-ment”, the speaking rate was calculated as 15/4.01=3.74 syllables/second.

### Pitch

The REAPER (https://github.com/google/REAPER) pitch estimator, was used to extract the pitch contour from the raw audio waveform for calculating the average pitch and pitch variance. The average pitch was estimated by calculating the sample mean from the pitch contour; similarly, the pitch variance was estimated by calculating the sample variance from the pitch contour.

### Vowel and Consonant Articulation Precision

The sentence reading audio files and corresponding sentence texts were estimated using the goodness of pronunciation (GOP) score evaluation algorithm to generate the vowel and consonant articulation precision scores.

### Vowel Space Area

All five sentence reading samples were concatenated into a continuous audio stream. The vowel space area was estimated using an extraction algorithm.

### Spontaneous Pause Rate

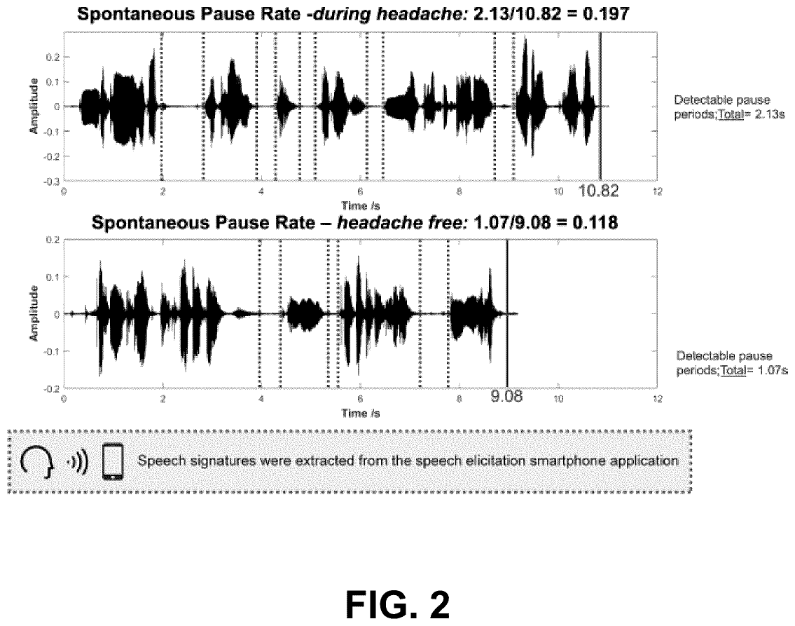

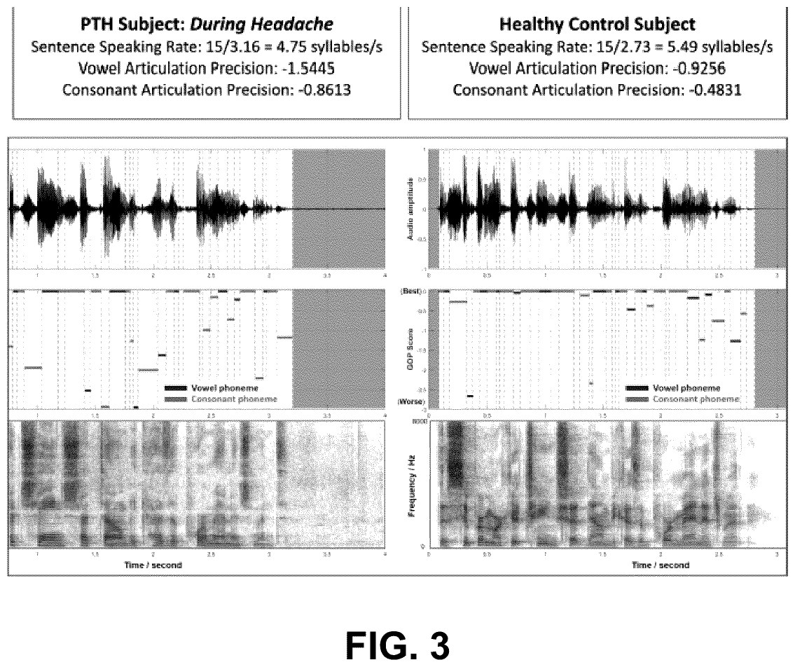

The VAD algorithm was used to detect the timepoints during which the participant was speaking. The total speaking time was measured as the period from the speech start point to the speech stop point; the pause time was measured as the non-speech periods during spontaneous speech. The spontaneous pause rate was then calculated as the ratio of pause time over speaking time. For example, if a subject provided a spontaneous speech sample lasting 10.82 s seconds, and paused for 2.13 s during the task, then the spontaneous pause rate was calculated as 2.13/10.82=0.197. The pause rate was measured from the spontaneous speaking task. (See FIG. 2 and FIG. 3)

Since some speech features may depend on the age and sex of the speaker (e.g., older people typically speak more slowly and in lower pitch; female speakers generally have higher average pitch compared to male speakers), feature normalization was used to control for these potential confounding variables. Speech features were normalized by subject age and sex using the Mozilla common voice English database, a large open-source corpus for speech data. This database contains sentence reading audio samples with corresponding texts for more than 11,000 individuals with age and sex demographics provided. Because it is a sentence reading database, only the features extracted from our sentence reading task are normalized, including sentence reading speaking rate, average pitch, pitch variance, and vowel and consonant articulation precision. Vowel space area is excluded from normalization because most individuals in the Mozilla database do not provide sufficient speech for computing this measure reliably. In addition, spontaneous pause rate is not normalized since there is no normative data for this task.

To normalize the features of a study participant, a nonparametric estimate of the cumulative distribution function (CDF) was computed from a subset of age/sex matched individuals in Mozilla. The features were converted to percentiles relative to this CDF and the normalized percentiles were then used as features.

### Statistical Approach

Speech patterns of subjects with PTH and healthy controls were compared using a mixed-effects model with random (unique) intercepts for each participant and controlled for age and sex. The effect of healthy controls/PTH was tested on each speech measure. Age, sex, and group differences were treated as fixed effects. Differences on cohort demographics were assessed via two-sided t-tests or Fisher-exact tests, as appropriate.

Speech patterns between subjects with PTH during headache were compared to speech patterns of subjects with PTH when they were headache-free using a mixed-effects model with random (unique) intercepts for each participant and random (unique) slope for headache status. Age, sex, and headache status were modeled using fixed effects. The use of random slopes tests not only whether there was a mean difference in the metrics when subjects had a headache compared to when they were headache-free but it also tests the extent to which participants differed on their changes on speech measures (e.g., some participants might have very different speaking rates when they have a headache as compared to when they are headache-free, while others may not change much).

Therefore, a significant p-value may indicate mean differences in a measure when headache is present versus absent or indicate that participants vary in terms of how their scores differ when they have a headache or are headache-free. Given the limited sample size, the models for some speech metrics did not converge. Non-convergence occurs when the model is too complex, the sample size is too small, or the model is not supported by the data, which results in the model being unable to reach a stable solution. Therefore, only the models that converged are reported.

### Results

Nineteen subjects with PTH (mean age=42.5, SD=13.7; 13 females, 6 males) and 31 healthy controls (mean age=38.7 SD=12.5, 18 females, 13 males) participated. There were no significant differences between groups for age (p=0.32) or sex (p=0.55); see Table 2, Subject Demographics.

TABLE 2
Subject Demographics.

| | PTH | | HC | | PTH vs HC |
|---|---|---|---|---|---|
| | (n = 19) | | (n = 31) | | p-value |
| Age, mean (SD) | 42.5 | (13.7) | 38.7 | (12.5) | 0.32 |
| Sex female/male | 13/6 | | 18/13 | | 0.55 |
| BDI mean (SD) | 7.63 | (5.6) | 2.06 | (3.5) | 0.001* |
| Symptom | 28.47 | (26.7) | 1.97 | (4.0) | <0.001 |
| Evaluation; total | | | | | |
| RAVLT delayed recall | −.90 | (1.1) | .15 | (1.2) | 0.004 |
| Average headache | 4.53 | (2.1) | n/a | | n/a |
| pain (1-10) | | | | | |

Average headache pain = ranges from 1 to 10 (1 = mild headache to 10 = worst headache imaginable) BDI = Beck Depression Inventory, mean raw score;
SCAT-3 = Sport concussion assessment tool (SCAT-5), Symptom Evaluation, total score;
RAVLT delayed recall = Ray Auditory Verbal learning test, delayed recall, z-score;
Days post-mTBI = number of days between the date of mild traumatic brain injury and the date of the baseline visit, on average, patients with PTH were seen two-weeks post-concussion (14.8 days, range 4-42 days);
HC = healthy controls;
n/a = not applicable;
PTH = Post-traumatic Headache

Among those with PTH, 9 had mTBI that were due to motor vehicle accidents, 3 that were due to falls, 5 that were due to sports-related injuries, and 2 that were due to hitting their head in home-related accidents. Fifteen subjects with PTH had no prior mTBI, 1 subject had one prior mTBI, and 3 subjects had two prior mTBIs. Twelve subjects reported no loss of consciousness, and 7 had loss of consciousness. As part of completing the headache questionnaire, individuals reported medication use for treating headache. Nine individuals reported treating headache with NSAIDs, and ten patients did not treat headache with medication. There were significant differences between groups on the symptom assessment of the SCAT-5, with individuals with PTH reporting more severe symptoms (symptom assessment total score: PTH=28.47; SD=26.7; healthy controls=1.97, SD=4.0; p<0.001) and they had significantly lower delayed recall z-scores compared to healthy controls (delayed recall: PTH=−0.9, SD=1.1; healthy controls=0.15, SD=1.2; p=0.004). Symptoms of aura including difficulty with speech was only reported by one individual with PTH. Although there were significant group differences on raw scores of the BDI, the mean raw scores of both groups were in the ‘normal, non-depressed’ range. On average, subjects with PTH were seen two-weeks post-mTBI (14.8 days, range 4-42 days).

A total of 1122 speech samples were collected (healthy controls=622; subjects with PTH=500; 180 samples of PTH during headache and 320 samples of PTH without headache).

Speech Differences Between Individuals with PTH and Healthy Controls

Regardless of headache presence or absence, individuals with PTH had significantly reduced consonant precision (not normalized: p=0.008; normalized: p=0.0015) and vowel precision (not normalized: p=0.007; normalized: p=0.0368) and longer pause rates (0.0098) relative to healthy controls. On days when PTH subjects had headache, subjects had significantly longer pause rates (p=0.0043), slower sentence speaking rates (not normalized: p=0.0369; normalized: p=0.0137) and less precise vowel (not normalized: p=0.049; normalized vowel articulation was not significant: p=0.1948) and consonant articulation (not normalized: p=0.0028; normalized: p=0.0038) compared to healthy controls.

Tables 3 and 4 show the speech measures, the p-values for the healthy controls/PTH differences (p-value computed based on a Chi Squared Likelihood Ratio Test), the mean values (e.g., mean pause rate) for the two groups, and the difference between the two groups. A significant p-value indicates that the mean speech measure differed significantly between the control and PTH groups.

The means and differences are based on the sample cohorts and not based on the mixed-effects model and are provided in order to give context to the p-value and to evaluate the directionality of the effect.

TABLE 3
Differences in speech patterns between
patients with PTH and Healthy Controls.

| Speech Measures | p-value | HC mean | PTH mean | diff |
|---|---|---|---|---|
| Consonant | 0.0008 | −0.8073 | −1.1144 | −0.307 |
| Articulation | | | | |
| Precision | | | | |
| Normalized | 0.0015 | 0.767 | 0.6172 | −0.1498 |
| Consonant | | | | |
| Articulation | | | | |
| Precision | | | | |
| Vowel | 0.007 | −0.9675 | −1.1779 | −0.2105 |
| Articulation | | | | |
| Precision | | | | |
| Spontaneous | 0.0098 | 0.1557 | 0.2029 | 0.0472 |
| Pause Rate | | | | |
| Normalized Vowel | 0.0368 | 0.6768 | 0.5565 | −0.1203 |
| Articulation | | | | |
| Precision | | | | |
| Vowel | 0.084 | 160158.442 | 143262.189 | −16896.254 |
| Space Area | | | | |
| Normalized | 0.2485 | 0.6845 | 0.6496 | −0.0349 |
| Speaking Rate | | | | |
| Sentence | 0.32 | 4.77 | 4.6157 | −0.1544 |
| Speaking Rate | | | | |
| Normalized | 0.5597 | 0.3063 | 0.2756 | −0.0307 |
| Average Pitch | | | | |
| Normalized | 0.5615 | 0.5576 | 0.552 | −0.0056 |
| Pitch Variance | | | | |
| Average Pitch | 0.7319 | 134.9549 | 145.7693 | 10.8144 |
| Pitch Variance | 0.9092 | 41.1519 | 45.3323 | 4.1804 |

diff = differences between the means of speech measures between PTH and HC, for assessing the direction of differences only. Negative numbers indicate lower values, and positive numbers indicate higher values for patients with PTH. Bolded p-values indicate significant differences between PTH and HC. Normalized = the value is normalized by a large corpus (for details refer to Table 1).

TABLE 4
Differences in speech patterns between
HC and PTH patients during headache.

| Speech Measures | p-value | HC mean | PTH mean | diff |
|---|---|---|---|---|
| Consonant | 0.0028 | −0.8073 | −1.127 | −0.3197 |
| Articulation | | | | |
| Precision | | | | |
| Normalized | 0.0038 | 0.767 | 0.6007 | −0.1664 |
| Consonant | | | | |
| Articulation | | | | |
| Precision | | | | |
| Spontaneous | 0.0043 | 0.1557 | 0.2057 | 0.05 |
| Pause Rate | | | | |
| Normalized | 0.0137 | 0.6845 | 0.5564 | −0.128 |
| Sentence | | | | |
| Speaking Rate | | | | |
| Vowel | 0.0302 | 160158.442 | 139356.524 | −20801.919 |
| Space Area | | | | |
| Sentence | 0.0369 | 4.77 | 4.2899 | −0.4802 |
| Speaking Rate | | | | |
| Vowel Articulation | 0.049 | −0.9675 | −1.1179 | −0.1505 |
| Precision | | | | |
| Normalized Vowel | 0.1948 | 0.6768 | 0.5887 | −0.0881 |
| Articulation | | | | |
| Precision | | | | |
| Average Pitch | 0.3975 | 134.9549 | 150.5934 | 15.6385 |
| Normalized | 0.6793 | 0.3063 | 0.3065 | 0.0002 |
| Average Pitch | | | | |
| Normalized | 0.6891 | 0.5576 | 0.5724 | 0.0148 |
| Pitch Variance | | | | |
| Pitch Variance | 0.9788 | 41.1519 | 46.3582 | 5.2063 |

Results of linear mixed model with individuals as random effects, controlled for age and sex.

Speech Differences in Individuals with PTH During Headache Compared to the Headache-Free State

During headache, PTH subjects had significantly slower sentence speaking rates (not normalized: p=0.002; normalized: p<0.0001) but more precise vowel articulation (normalized: p=0.0052) compared to when they were headache-free. Table 5 shows the speech measures, the p-values for the differences between the headache states, the mean values for the two headache states, and the mean differences between the two headache states. The means and differences are based on the raw scores of the sample and not based on the mixed-effects model and are provided in order to give context to the p-values and to evaluate the directionality of the effect. Two sets of p-values are provided: p-values for the random-intercepts models and p-values for random-intercepts-random-slopes models. As previously explained, significant p-value in the random-intercepts model indicates that the mean speech measure differed significantly between the headache states, while a significant p-value in the random-intercepts-random-slopes indicates significant mean differences and between-participant variability in the differences between the two states.

TABLE 5
Speech pattern differences in patients with PTH during days of headache
(headache ‘yes’) compared to days of headache freedom (headache ‘no’).

| | p-value | p-value | | | |
|---|---|---|---|---|---|
| | (random-intercepts- | (random- | | | |
| | random-slopes | intercepts | Headache | Headache | |
| Speech Measures | model) | model) | ‘no’ | ‘yes’ | diff |
| Normalized | <0.0001 | 0.0003 | 0.7866 | 0.5659 | 0.2206 |
| Sentence | | | | | |
| Speaking | | | | | |
| Rate | | | | | |
| Sentence | 0.0020 | 0.0036 | 5.1108 | 4.2128 | 0.8981 |
| Speaking | | | | | |
| Rate | | | | | |
| Normalized | 0.0052 | 0.0034 | 0.4658 | 0.5798 | −0.114 |
| Vowel | | | | | |
| Articulation | | | | | |
| Precision | | | | | |

Results of linear mixed model with individuals as random effects controlled for age and sex. Random-intercepts model = Bolded p-values indicate that the mean of the speech measure is different when the participant has a headache as compared when she/he is headache free (within-person effect). Random-intercepts-random-slopes model = Bolded p-values indicate that there were differences not only on the mean of the speech measures between the headache states but also on how participant differed on their changes on speech measures between the two headache states (within- and between- person effects).

### DISCUSSION

The results of this study demonstrate longer pause rates, slower sentence speaking rates and less precise vowel and consonant articulation in patients with PTH during headache compared to healthy controls as well as slower sentence speaking rates and altered vowel articulation in individuals with PTH during headache as compared to when PTH subjects were headache-free.

Our results are in agreement with previous migraine and chronic pain studies which identified slower motor speech production (speech alternating motion rates) in individuals with chronic back pain and changes in speaking rate, articulation rate, articulatory precision, phonatory duration, and intonation shown between individuals with migraine relative to healthy controls as well as within the migraine group during the pre-attack vs. attack vs. interictal periods [4]. Although it was not the focus of the current study, it is important to note that psycholinguistic changes are also observed in patients suffering from psychological trauma, such as post-traumatic stress disorder [20] and childhood trauma. Although these disorders are difficult to disentangle (i.e., PTSD and PTH due to TBI often co-occur), future studies are needed to assess the similarities and unique differences of speech alterations in patients with PTH from speech alterations in individuals with PTSD from changes in speech in children suffering emotional distress due to traumatic life experiences.

### Speech Rate and Rhythm (Spontaneous Pause Rate and Sentence Speaking Rate)

Compared to healthy controls, individuals with acute PTH demonstrated alterations in speech rate and rhythm (i.e., longer pause rates and slower sentence speaking rates). There is emerging data that individuals with PTH have difficulty understanding and performing cognitive-linguistics tasks, have difficulty understanding and processing rapid speech and show electrophysiological evidence of abnormal auditory processing. Saunders et al., found that blast-exposed veterans with mTBI had auditory processing deficits despite having clinically normal hearing, and Strockbridge and colleagues found that concussed children had altered language profiles and difficulty with semantic and syntactic access relative to non-concussed healthy children. Furthermore, a recent study by Talkar et al. showed that vocal acoustic features of articulation, phonation and respiration can distinguish individuals with subclinical mTBI from healthy controls.

Although not the focus of this study, participants with acute PTH did show significantly worse performance on a delayed word recall task (RAVLT, delayed recall) and more cognitive, behavioral and mood related symptoms (SCAT-5). Therefore, pause rates in individuals with PTH could be an indication of word-finding difficulties and may serve as a proxy for cognitive function in individuals with PTH. However, future studies are needed that specifically relate post-mTBI symptoms including cognitive function to changes in speech.

### Precision of Articulation (Vowel Space Area, Vowel and Consonant Articulation Precision)

In the current study, individuals with PTH during headache also showed alterations in the precision of articulation, specifically reduced vowel space area relative to healthy controls.

Vowel space area is an acoustic metric commonly used for measuring articulatory function. Previous data have shown reduced vowel space area in patients with motor speech disorders including Parkinson's disease and cerebral palsy as well as in patients with depression and those suffering from post-traumatic stress disorder and vowel space area has thus been suggested as a potential marker for psychological distress. In the current study, subjects had depression symptoms within normal/healthy range, therefore it is anticipated that reduced vowel space area may be a manifestation of either speech production under stress (i.e., headache pain intensity) or related to difficulties with speech-motor control due to the underlying mTBI. This hypothesis is further supported by the reductions in both vowel and consonant precision in patients with PTH relative to healthy controls and the reduction in speaking rate between the PTH group and the HC group and between PTH during headache vs when individuals with PTH were headache free.

The disruption in speech pattern in subjects with PTH might be a result of brain structural or functional changes in auditory and language pathways such as the posterior thalamic fasciculus and the superior and inferior longitudinal fasciculus. However, the neural underpinnings of speech changes will need to be further interrogated by associating brain structural and functional data with speech features in subjects with PTH.

Individuals with PTH had more precise vowel articulation during headache compared to when they were headache-free. It may be hypothesized that during headache, when speech production requires more effort (hence resulting in slower speaking rates), individuals need to pay more attention to the production of speech and thus paradoxically produce more precise vowel articulation.

It is possible that several factors may have influenced individuals' speech patterns and introduced variance to our result including 1) mTBI mechanism (sports-related vs, motor vehicle accident vs fall), or 2) the number of previous mTBIs. Assessment of speech features in subjects with mTBI without headache to subjects with PTH can specifically disentangle speech changes due to mTBI from speech changes due to headache. Additionally, studies are contemplated to isolate speech alterations in individuals with PTH without history of PTSD from individuals who suffer from PTSD without history of mTBI. In the current study, the model for pause rate did not converge in the within-subject analysis which is likely due to the relatively small sample size in the study. It is anticipated that a larger study, with more speech samples captured during periods of headache and no-headache per individual, would further show that reductions in pause rate are apparent in the within-subject analysis as well.

### CONCLUSION

The results indicated changes in speech rate and rhythm and alterations in precision of articulation in individuals with PTH to due mTBI relative to healthy controls as well as a reduction in sentence speaking rate and alterations in vowel articulation precision when individuals with PTH had a headache compared to when they were headache-free—potentially suggesting that PTH-related pain can modify healthy speech patterns. Currently, there is not a way to predict when and whether an individual with PTH will recover. The current results indicate that speech detection using a speech application downloaded on a mobile device might be a practical, objective, and early rapid screening tool for assessing headache-related burden and may have potential for predicting headache recovery in subjects with acute PTH. Additionally, the recognition of speech changes in individuals with acute PTH could be important for identifying those individuals at ‘high risk’ for developing persistent post-traumatic headache and may allow physicians to begin headache treatment early, when it might be most effective, in order to prevent headache chronification.

### Key Findings

Relative to healthy controls, individuals with acute PTH show aberrations in objective speech features.

Speech changes are exacerbated in PTH subjects during headache.

Speech pattern analysis has utility for assessing headache burden and recovery.

### Prognostic Biomarker Signature to Predict the Persistence of Post-Traumatic Headache

Over 2 million people are diagnosed with concussions each year, with approximately 300,000 concussions resulting from sports-related activities alone. From 2000 to 2018, approximately 380,000 traumatic brain injuries (TBIs) were reported amongst U.S. military personnel and over 80% of these were mild TBI's (mTBI), which are often referred to as concussions. The consequences of concussion due to post-concussion symptoms, treatment side effects, and the resulting disability, place a staggering burden on individuals and society. In the military alone, the cost of care has risen from $21 million (2003) to $646 million (2010).

According to recent guidelines, a concussion is defined as an event caused by a sudden blow to the head or other part of the body resulting in a short-term disturbance of neurologic function. One of the most common symptoms immediately following a concussive injury is post-traumatic headache (PTH). Although acute PTHs can resolve over the course of a few days, a significant proportion of concussed patients with PTH have persistence of PTH (PPTH), which is classified according to the International Classification of Headache Disorders (ICHD-III) diagnostic guidelines as headaches that persist for 3 months or longer from onset. Those individuals who develop PPTH are at significantly higher risk for pain-related disability and for seeking medical care post-injury compared to injured patients without PPTH. Furthermore, once PPTH develops it is often quite refractory to treatment.

Although concussion can manifest with a myriad of short-term and long-term symptoms that can be quite severe, routine diagnostic brain imaging is unable to detect subtle anatomical abnormalities associated with concussion or PPTH. Furthermore, there is currently no accurate way of predicting whether a patient with PTH will recover quickly (and become headache-free within three months of PTH onset) or will have PPTH that may continue over many months to years. This inability to predict who will recover quickly and who will have PPTH is a current patient care dilemma that prevents a clinician from making knowledgeable decisions regarding early treatment of PTH and from prognosticating recovery. The development of a prognostic biomarker signature for identifying patients at high risk for PPTH is an unmet need that would allow clinicians to administer more timely non-opioid pharmacological and non-pharmacological therapy to those in need, with the aim of preventing PTH persistence. Furthermore, identifying patients that are highly likely to recover on their own could prevent unnecessary medical treatment (and associated side effects and toxicities) and unnecessary follow-up care. Lastly, the identification of patients who are highly likely to have PTH persistence would be very useful for future clinical trials of PTH non-opioid treatments since it would be a means of enriching subject populations with patients who are otherwise highly likely to have PTH persistence. In some embodiments, a prognostic biomarker signature for PPTH is developed by fusion of brain imaging data with in-depth clinical data collected from questionnaires, simple cognitive tests, and a speech assessment paradigm.

Advanced structural and functional analyses of brain magnetic resonance imaging (MRI) data provide better sensitivity for detecting anatomical and functional changes associated with concussion and PTH than routine neuroimaging techniques. An increasing number of studies have found changes in volume and cortical thickness in patients during the acute and chronic stages of concussion, and our own work demonstrates similar findings in patients with PTH. Additionally, diffusion tensor imaging (DTI), which allows for the interrogation of axonal injury by assessing the diffusivity of water along white matter, and resting-state functional MRI, which measures the functional connectivity of the brain, are valuable tools for measuring immediate structural and functional brain changes following concussion. This study aims to (a) identify the semi-acute effects of concussion and PTH on brain structure and function and to (b) predict, using prognostic models based on neuroimaging and in-depth clinical data, which person is going to recover from PTH during the acute phase versus which person is likely to have PTH persistence.

### Preliminary Data

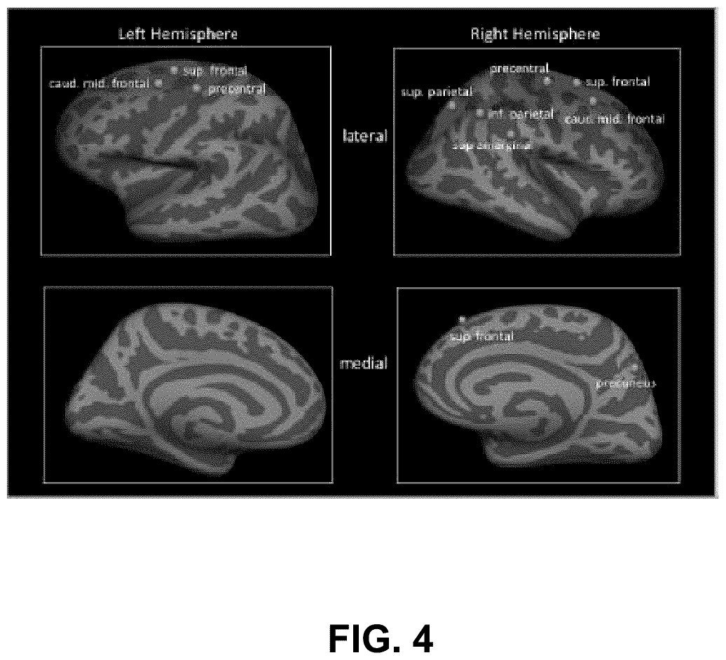

Preliminary data has been collected from individuals with concussion that demonstrate regions of less cortical thickness (shown by the color ‘red’ on FIG. 4) in patients with PPTH relative to healthy controls, white matter tract alterations in patients with semi-acute PTH and PPTH relative to healthy controls, and differences in brain structure between patients with migraine vs. PPTH using T1-weighted imaging and DTI. Additionally, there are published findings using T1-weighted and resting-state functional magnetic resonance imaging (rs-fMRI) data demonstrating the utility of both structural and functional neuroimaging data for distinguishing individual patients with migraine from individual healthy controls; similar methodology will be used in this PTH research and interrogates the utility of structural and functional neuroimaging methods, in-depth clinical data and speech signatures for discriminating between patients at high risk for PPTH and patients who will recover from PTH during the acute phase.

### White Matter Fibertract Alterations in Acute PTH

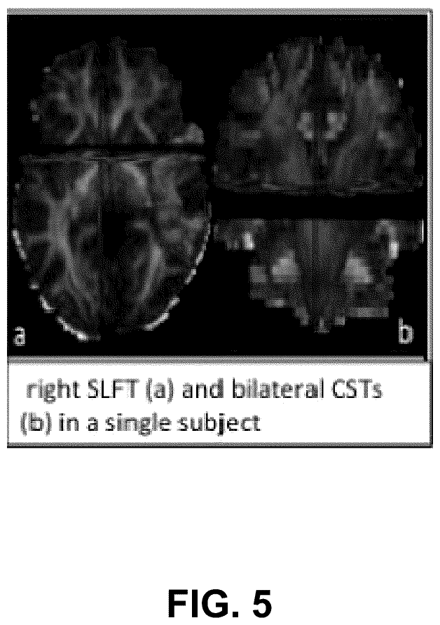

A DTI analysis for 5 male patients with PTH (<12 weeks post-concussion) and 5 male, age-matched, healthy controls. Mean diffusivity of the cortico-spinal tract (CST) and the superior longitudinal fasciculi (SLFT) were examined, as both tracts have been previously implicated as being vulnerable to concussion. Results revealed significant group differences for the bilateral SLFT and the CST. For both tracts, concussed patients showed increased mean diffusivity, suggesting edema and acute tract damage (see FIG. 5). The data from the experiment is displayed in Table 6. Results were encouraging and indicated that tract specific fiber tract damage can be detected in patients with acute PTH.

TABLE 6

| | PTH patients | Healthy controls | |
|---|---|---|---|
| Fibertract MD | n = 5 | n = 5 | p-value |
| R SLFT | .00074(.000011) | .00071(.000019) | .013 |
| L SLFT | .00072(.000015) | .00070(.000019) | .048 |
| R CST | .00075(.000024) | .00071(.000017) | .047 |
| L CST | .00074(.000023) | .00071(.000017) | .021 |

MD = Mean Diffusion; L = Left; R = Right; SLFT = Superior Longitudinal Fasciculus; CST = Corticospinal tract; Standard deviation shown in parentheses.

### White Matter Fibertract Alterations in PPTH

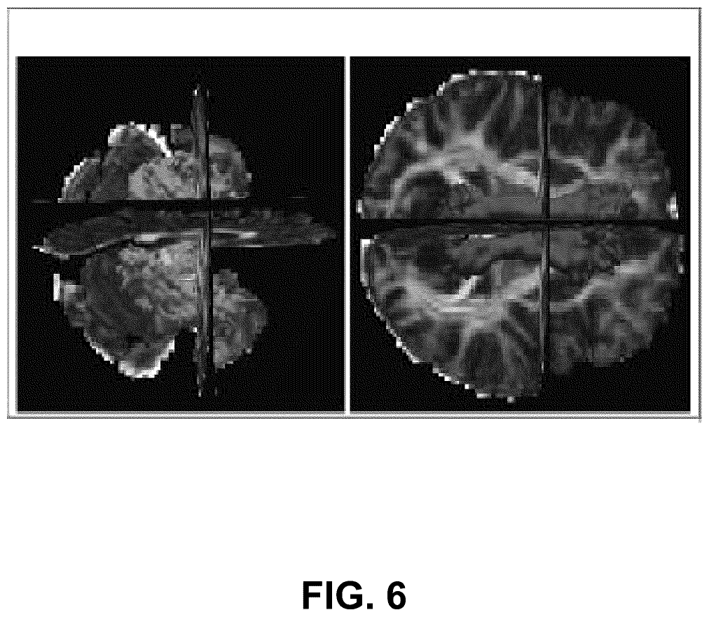

White matter fibertract patterns were compared in PPTH (n=49) relative to healthy controls (n=41). PPTH had stronger mean diffusivity and radial diffusivity in the bilateral cingulum tracts (angular bundles and cingulate gyri) (see FIG. 6). In PPTH there was a significant positive correlation between headache frequency and bilateral cingulate angular bundle mean diffusivity and radial diffusivity (p<0.05), suggesting that headache frequency might modulate the integrity of the cingulum white matter fibertracts (Chong et al, Cephalalgia, 2018).

### Cortical Thickness Findings in PPTH

The purpose of this analysis was to investigate differences in cortical thickness and white matter integrity in patients with PPTH relative to healthy controls and to interrogate whether cortical morphology relates to headache burden in patients with PPTH. Patients with PPTH had less cortical thickness relative to healthy controls in the left and right superior frontal, caudal middle frontal and precentral cortex as well as less cortical thickness in the right supramarginal, right superior and inferior parietal and right precuneus regions (p<0.05, Monte Carlo corrected for multiple comparisons). There were no regions where patients with PPTH had more cortical thickness relative to healthy controls. There was a negative correlation between left and right superior frontal thickness with headache frequency (p<0.05), potentially indicating that brain morphology changes in the superior frontal regions in patients with PPTH are modified by headache frequency.

### Brain Structural Differences in PPTH Vs Migraine

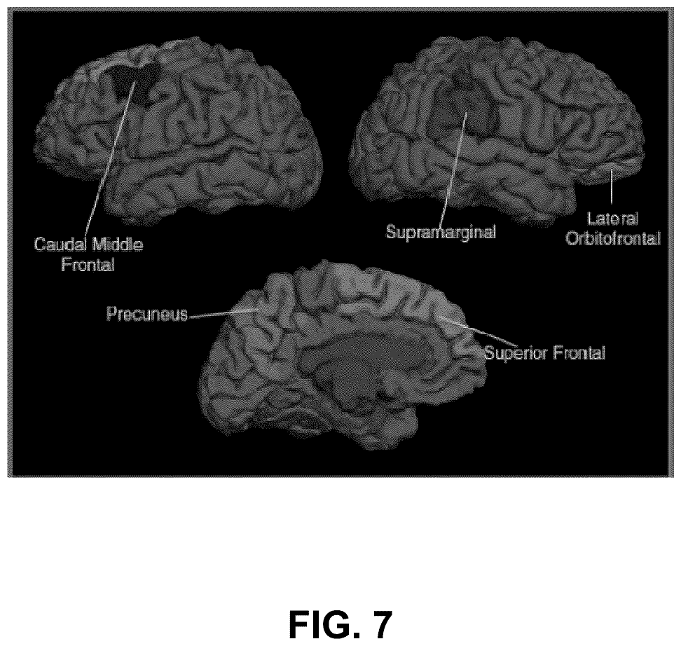

The purpose of this study was to identify changes in brain structure (cortical thickness, volume, surface area and brain curvature) in patients with PPTH compared to patients with migraine. Differences in cortical thickness were compared between subject groups using an ANCOVA design. There were several brain regions that showed differences in brain structure between migraine and PPTH, including the right lateral orbitofrontal lobe, left caudal middle frontal lobe, left superior frontal lobe, left precuneus and right supramarginal gyrus (see FIG. 7). This study demonstrates that patients with migraine and patients with PPTH have differences in brain structure in frontal and parietal regions, suggesting differences in the neuropathology between the headache types.

### Relationship Between Functional Connectivity Patterns and Symptom Recovery Following Concussion—Preliminary Longitudinal Data

Functional connectivity patterns were interrogated in 15 concussed patients and 15 healthy controls. Resting-state MRI was used to estimate homotopic functional connectivity patterns in 29 predefined regions. Concussed patients underwent imaging at two time-points; semi-acutely following concussion and 4-months post time-point 1. Results indicate weakening of homotopic region connectivity in semi-acutely concussed patients relative to healthy controls (HC) in the primary somatosensory area, spinal trigeminal nucleus, middle cingulate and the posterior insula. At 4-months follow-up homotopic region connectivity showed a trend toward normalization. For concussed patients, there was a significant negative correlation between somatosensory functional connectivity strengthening (from the semi-acute phase to the 4-month follow-up) and symptom severity potentially suggesting that functional strengthening might be associated with post-concussion symptom recovery.

Knowledge Gained from Preliminary Data for Patients with Acute PTH and PPTH

In summary, the preliminary data indicate that patients with acute PTH and PPTH due to concussion have higher mean and radial diffusivity for specific fibertracts known to be vulnerable to head trauma and less cortical thickness over widespread frontal regions. Structural MRI and DTI data indicate a relationship between headache burden and cortical and white matter integrity—potentially indicating the utility of DTI and structural MRI for longitudinally tracking brain neuropathology following concussion. Furthermore, patients with PPTH have changes in cortical structure compared to patients with migraine demonstrating the use of structural brain imaging for identifying differences between phenotypically similar headache types and potentially for sub-classifying individual headache types. Lastly, preliminary longitudinal data suggest a relationship between functional connectivity patterns and symptom recovery following concussion, indicating the use of functional MRI for tracking symptom relief.

### Classification Models Based on Brain Structural Data

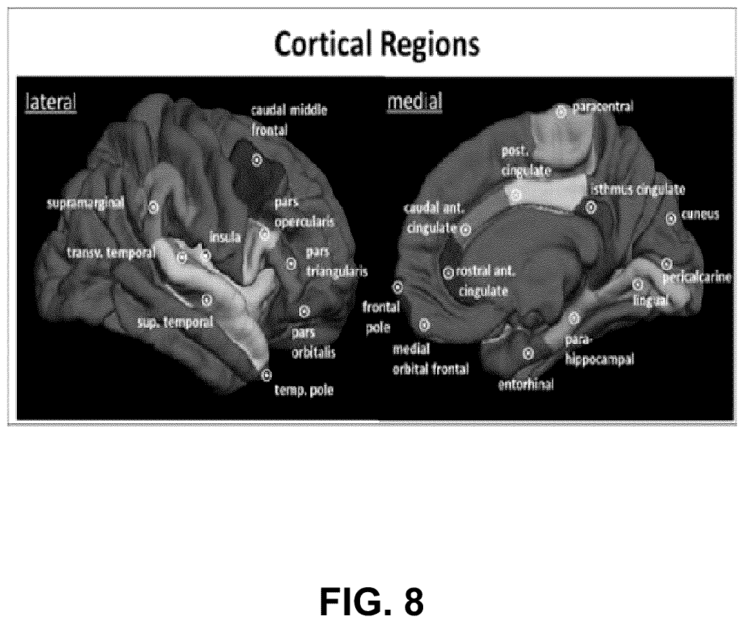

Work has been published developing a classification model based on structural data for distinguishing chronic migraine patients from healthy controls. Using FreeSurfer version 5.3, T1-weighted scans were automatically parcellated into regional measures of cortical thickness, volume and area (see FIG. 8). These regional estimates from 66 migraine patients and 54 healthy controls were used as input for a principal component analysis followed by a classification algorithm to distinguish between individual migraine patients and individual healthy control subjects. Using a ten-fold cross validation approach, the data were split into 10 subgroups, so that data-points used to train the classification algorithm were always different from the data-points that were used to test the classification algorithm.

The efficacy of several classification algorithms were tested including diagonal linear discriminate analysis (DLDA), diagonal quadratic discriminate analysis (DQDA), support vector machine (SVM), and decision tree (DT), and it was found that DQDA produced the best classification results. Results indicated that principal components consisting of structural measures from the temporal pole, anterior cingulate cortex, superior temporal lobe, entorhinal cortex, medial orbital frontal gyrus, and pars triangularis best distinguished individual chronic migraine patients from individual healthy controls (i.e. classified a single brain MRI as belonging to someone with chronic migraine vs. belonging to a healthy control) with an average accuracy (over 10 runs) of 86.3% and a best accuracy (best of 10 runs) of 88.6%. shows important regions over the left and right hemisphere that best distinguished chronic migraine patients from healthy controls.

### Classification Models Based on Brain Functional Connectivity Data

There have been published findings using resting-state functional connectivity (rs-fc) data for distinguishing migraine patients from healthy controls. 33 regions of interest (ROIs) were selected with known importance for sensory processing and interrogated the functional connectivity patterns of these ROIs with every other voxel in the brain. As this produced over 50,000 individual data points, a data reduction algorithm (principal component analysis) combined with a forward stepwise search was used for searching the data to determine those components that would best distinguish individual migraine patients from healthy controls. These components were then used to train a DQDA classifier. Results indicated that 8 out of 10 migraineurs could be accurately classified based on the functional connectivity patterns of the bilateral amygdala, the right middle temporal, right posterior insula and right middle cingulate as well as the left ventromedial prefrontal cortex.

Changes in Speech Production Resulting from TBI and Migraine

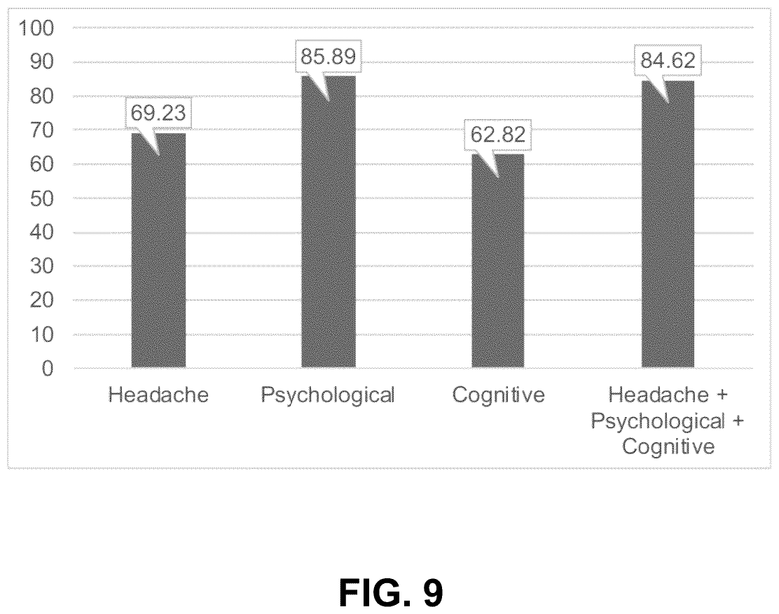

The relationship between head trauma and changes in speech production has been studied in different sports-related cohorts. In a recently-published paper it was demonstrated that at-home monitoring of speech and language through a dedicated mobile app can predict the signs of an oncoming migraine attack in individuals without aura as early as 12 hours prior to onset. Briefly, a total of 56,767 speech samples were collected, including 43,102 from 15 individuals with migraine and 13,665 from matched healthy controls. Significant group-level differences in speech features were identified between those with migraine and healthy controls and within the migraine group during the pre-attack vs. attack vs. interictal periods (all p<0.05). Most consistently, speech changes occurred in the speaking rate, articulation rate, articulatory precision, phonatory duration, and intonation. Within subject analysis revealed that seven of 15 individuals with migraine showed significant change in at least one speech feature when comparing the migraine attack vs. interictal phase and four showed similar changes when comparing the pre-attack vs. interictal phases. FIG. 9 shows a comparison of the cohort with headache, psychological, cognitive, and headache+psychological+cognitive symptoms.

### Classifying Between Migraine and PTH Using Clinical Data

In ongoing work, development of classifiers that discriminate between patients with Migraine and those with PTH will be performed. In our first set of experiments, a developed models was used to assess which clinical measures classify between Migraine and PTH cohorts. The clinical measures were first split into three categories: Headache Measures, Psychological Measures, and Cognitive Measures. A linear logistic ridge regression classifiers was trained for each of three modalities separately and in combination. The ridge parameter is set using a held-out development set and the performance of the model is evaluated on a held-out test set. Due to the limited sample size, a leave-one-out cross-validation approach to both the development set and the held-out test set was used. It is clear that while both the Headache and Cognitive clinical features provide some predictive power when it comes to the task (69.23% and 62.82% respectively), the psychological clinical features yield the highest performance with an accuracy rate of 85.89%. When combined across the three modalities, the performance is close to the model that uses Psychological features only (84.62% vs. 85.89%).

For each of the classifiers described above, the most informative features in the classifier were also analyzed. It is interesting to note that, when combined across all three modalities, the psychological features (namely those that measure PTSD symptoms) are most predictive of PTH. This explains the similarity in the performance of the model that only uses psychological features and the model that combines across the three modalities.

Knowledge Gained from Classification Studies

Testing of algorithms that provide the best accuracy for distinguishing between patients and controls. DQDA was identified as providing the best accuracy. DQDA will be used as well as interrogate the accuracy of other algorithms for the predictive modeling of patients with PPTH.

Development of classification models based on brain volume, thickness and area and speech signature data that have potential for classifying individual patients from individual controls. A similar techniques will be sued to build classification models in our current proposal to predict which individual patients will have persistence of PPTH vs. recovery of PTH during the acute phase.

Data reduction algorithms are useful for ‘pruning’ rs-fc data into meaningful components. These data reduction algorithms will be used to determine those features that substantially contribute to the prognostication of PPTH.

Based on these previous findings, PTH patients may show altered connectivity amongst these 33 ROI relative to healthy controls and these functional alterations are expected to be exacerbated in those concussed patients that develop PPTH.

Preliminary results suggest that classification models based on brain structural data, rs-fc data and speech signature data have good utility for classifying individual patients from healthy controls and support the applicability of rs-fMRI, structural, and clinical data for developing prognostic biomarkers for PTH.

### Background of Future Study

There is currently no recognized way of accurately predicting who will recover from PTH during the acute phase following concussion and who will go on to develop PPTH, a condition that is difficult to treat effectively. Over the course of the R61 period, brain imaging and clinical feature biomarkers will be identified using machine-learning algorithms that distinguish individuals at high risk for developing PPTH from patients who are likely to acutely recover from PTH prior to three months. Results of this study will determine important clinical factors and neuropathological mechanisms underlying PPTH and will interrogate the contribution of clinical, demographic data, and speech signatures on predicting PTH persistence. Additionally, these results will determine the relative predictive weight of specific clinical factors and neuroimaging features for prognosticating which individuals are at higher risk for developing PPTH.

### Overview

The objective of this study is to use a machine-learning approach to identify individual patients with acute PTH who are at high risk for persistence of PTH, based on clinical data and structural and functional neuroimaging findings collected (7-28 days) since onset of PTH. This study aims to identify a prognostic biomarker signature associated with PPTH by assessing brain structure features (gray matter volume, area and thickness and white matter tract integrity), brain function features (functional connectivity, brain perfusion), and detailed clinical data collected using a comprehensive headache symptom battery, in patients with acute PTH.

To assess clinical characteristics and symptoms, and brain MRI structural and functional alterations in 100 semi-acutely concussed patients with PTH. Patients who develop PPTH will be compared to patients who recover during the acute phase of PTH and compared to 50 healthy controls. This will identify clinical characteristics and symptoms, and structural and functional brain changes that associate with the persistence of PTH. These results will inform the development of biomarker signatures in Aim 2 and Aim 3.

It is expected that MR imaging completed between 7-28 days since onset of PTH will show greater magnitude of alterations and greater distribution of changes in brain structure and function in patients that go on to have persistence of PTH compared to concussed patients that recover from PTH during the acute phase and compared to healthy controls. More specifically, the following findings in structural measures are expected to be found. Previous studies have shown less cortical thickness and less volume and surface area following mTBI and in association with posttraumatic symptoms including PTH and a relationship between changes in cortical structure and headache patterns in PPTH. It is expected that when evaluated during the semi-acute phase, patients who eventually are found to have persistence of PTH will have less cortical thickness, volumes, and surface area in regions related to pain processing and multisensory integration including regions in the anterior insula, posterior insula, anterior cingulate cortex, thalamus, somatosensory cortex, temporal-parietal junction, and parietal-occipital junction. Immediately following mTBI and in the acute PTH phase, increased fractional anisotropy (FA), mean diffusivity (MD) and radial diffusivity (RD) is shown to indicate white matter tract damage. It is expected that patients who eventually develop PPTH will have greater increases in measures of FA, MD and RD when measured semi-acutely following concussion and that these alterations in diffusion measures will be identified within longer anterior-posterior tracts such as the superior and inferior longitudinal fasciculi and the thalamic radiations. The following findings in functional measures are expected to be found. Based on previous findings, it is expected that during the semi-acute phase, individuals who eventually have persistence of PTH will have greater and more widespread alterations, relative to those who have resolution of PTH during the acute phase, in functional connectivity amongst regions that participate in different aspects of pain processing, pain modulation, and multisensory integration including: anterior and posterior cingulate regions, superior frontal areas, temporal pole, supramarginal, and limbic regions. Based on results of previous publications, it is expected that past history of concussion, presence of post-traumatic stress disorder, female sex, and history of headaches prior to concussion will increase the likelihood of developing post-traumatic symptoms, including PPTH. It is expected that the modality of injury (MVA vs sports-related) as well as alterations in speech signatures following concussion will contribute to predicting patients with PPTH. Although a limited number of studies have had some success in using clinical information for predicting persistent post-concussive symptoms, there is currently no known way of predicting who will develop PPTH based on clinical information alone. Thus, in-depth clinical features will be combined with MRI features to develop an accurate predictive model for PPTH while determining the relative contribution of clinical and imaging data. Table 7 shows a chart with an example of brain imaging acquisition parameters, according to some embodiments.

TABLE 7

| Siemens | Technical | Acquisition |
|---|---|---|
| Skyra 3T MRI | Details | Time |
| T1 3D | FOV: 256 × 256 mm² | 5.35 min |
| MPRAGE | Thickness: 1.25 mm; TR: 2400 ms; | |
| | TE: 3.03 ms; Voxel size: 1. × | |
| | 1. × 1.3 mm³ | |
| T2 Turbo | FOV: 256 × 256 mm², | 3.08 min |
| Spin Echo | Thickness: 4.0 mm; TR: 6800 ms; | |
| | TE: 84 ms; Voxel size: 1. × 1. × 4 mm³ | |
| DT1 | FOV: 220 × 220 mm², Thickness: 4.0 | 5.33 min |
| | mm; TR: 5100 ms; TE: 71 ms; | |
| | Voxel size: 1.7 × 1.7 × | |
| | 4 mm³; 60 directions | |
| Arterial Spin | FOV: 64 × 62 mm², Thickness: 5 mm; | 5.09 min |
| Labeling (ASL) | TR: 4600 ms; TE: 22 ms; | |
| | Voxel Size: 1.7 × 1.7 × 5 mm³ | |
| BOLD Resting- | FOV: 256 × 256 mm², | 10.00 min |
| State | Thickness: 4 mm; | Total Time: |
| | TR: 2500 ms; TE: 27 ms; | 29.40 min |
| | Voxel size: 4. × 4. × 4 mm³ | |

### Research Strategy

100 subjects with PTH and 50 healthy controls balanced to the cohort of PTH patients for age and sex over 30 months of active enrollment. All patients will be adults (18-65 years of age). PTH and PPTH will be diagnosed using the ICHD-3 diagnostic criteria for PTH attributed to mTBI (concussion). Exclusion criteria for healthy controls and patients with PTH include: 1) history of moderate or severe TBI, 2) prior history of gross anatomical change on imaging, 3) contraindication to MRI, including but not limited to severe claustrophobia and/or presence of ferrous materials in the body, 4) women who are pregnant, or believe that they might be pregnant. Although there are no known contraindications or risks associated with pregnancy and MRI, pregnant women or women who believe that there might be a chance that they are pregnant will be excluded. For patients with PTH, only patients with new onset of PTH without history of PPTH will be included in the study. A personal history of prior concussion and history of migraine are allowed according to ICHD-III diagnostic criteria. Additional exclusion criteria for healthy controls include history of concussion or more severe TBI, and history of migraine or other headaches. Tension-type headaches on three or fewer days per month is allowed for healthy control subjects. The diagnosis for PTH and PPTH will be verified by a board-certified physician in Neurology and Headache Medicine. Presence of concussion will be verified using the Ohio State University TBI Identification Method, a standardized questionnaire assessing the lifetime history of TBI for an individual. This method of identifying TBI is based on definitions and recommendations from the Center for Disease Control and Prevention.

All subjects with PTH will be prospectively enrolled for two appointments and reimbursed for their time. Transportation fares will be provided for subjects who do not have means of transportation or are unable to drive. The first appointment will occur 7-28 days since onset of PTH (time-point I) and will include a comprehensive Headache Symptom Battery and a brain MRI. The second appointment will be scheduled 12 weeks post time-point I testing (time-point II) and will include only the Headache Symptom Battery. An illustrative example of a symptom battery for post-traumatic brain injury, according to some embodiments is shown in Table 8. Data collected during this follow-up visit will be used to determine who has PPTH and who has already recovered from PTH.

TABLE 8
Symptom Battery

OSU TBI-ID
Insomnia Severity Index
PAQ
ASC-12
SCAT 5
Hyperacusis questionnaire
MIDAS questionnaire
COMPASS-31
PCL-5 checklist
NSI and OISS
Detailed headache symptoms assessment
Modified CDE forms
Trails A and B
RAVLT
STATE/TRAIT
BDI
Speed assessment
Time to complete: 60 min

Healthy controls will undergo neuroimaging and will complete questionnaires that assess demographic information and behavioral and headache characteristics at baseline and at a 12 week follow-up visit to ensure that healthy controls still have less than 3 tension-type headaches per month and did not have a new diagnosis of migraine. Healthy controls that develop migraine or have had more than 3 tension type headaches per month will be excluded from the analysis. 100 male and female subjects with PTH and 50 healthy controls will be assessed. Groups of healthy controls and groups of patients with PTH will be balanced for age and sex. Concussed subjects with PTH at time-point II testing will be diagnosed with PPTH, using ICHD-III guidelines. It is expected that 10% of patient imaging data will be ‘unusable’ either due to movement in the scanner, or scanner-related acquisition errors, due to patient attrition, i.e. patients not following through with their follow-up appointment or identification of gross anatomical abnormalities on MRI.

### Sample Size Determination and Power Analysis

For determination of sample sizes, it will be assumed that about 50% of individuals with acute PTH that has lasted for 7-28 days will go on to have PPTH. Of note, if individuals are enrolled earlier after onset of PTH (i.e. within the first week), the rate of PTH persistence would be much lower. For estimating group differences for functional imaging data, study sample size was determined by calculating the number of subjects needed per group to detect a difference in correlation coefficient of 0.1 in our rs-fc MRI design, based upon published data and assuming a standard deviation of 0.15 for functional connectivity correlation coefficient change. Based on a type I error of 0.05, sample sizes of 50 subjects in the PPTH cohort and 50 subjects who resolved in the acute phase of PTH would yield 90% power to detect pairwise rs-fc group differences. There is potential that there will end up being unequal subject cohorts. If it is assumed that there will be unequal sample sizes (not distributed 50/50) of patients with PTH versus PPTH there will still be enough power to detect group differences with a 40/60 distribution (89.9% power) or a 30/70 distribution (85.7% power). In predictive models, a power analysis is based on Area Under the Curve (AUC), which is an accuracy metric for a classifier. AUC is between 0 and 1 and with bigger AUC indicating higher accuracy.

Methods to assess brain MRI structural and functional alterations in individuals who eventually develop PPTH compared to those who recover from acute PTH. This aim will assess the semi-acute effects of TBI and PTH on brain structure and function and identify semi-acute changes that associate with the persistence of PPTH. The healthy control cohort will inform the direction of brain change relative to the PTH cohort.

### Neuroimaging and Post-Concussion Clinical Assessment

Imaging and the completion of the detailed clinical Headache Symptom Battery at time-point I will take approximately 2 hours. Completion of the Battery at time-point II will take approximately 1 hour. The neuroimaging protocol will include a 3D DTI 30-direction imaging sequence, a 3D high-resolution T1 scan, a T2 scan, an arterial spin labeling (ASL) sequence and a functional MRI blood-oxygen-level-dependent (BOLD) sequence. All scanning will be conducted on a 3Tesla machine at Mayo Clinic. These scanning sequences will take little time to acquire, do not require administration of contrast material, and do not require patients to participate in any task during the scan, characteristics that could allow for inclusion of all of these sequences during routine clinical MRI scans.

The comprehensive Headache Symptom Battery will include the following to assess physical symptoms of concussion and symptoms associated with headache: The Ohio State TBI identification questionnaire will be used to determine detailed information on the concussion history including number of prior concussions, modality of concussions, symptoms following concussions etc. (OSU TBI-ID; Available at: www.brainline.org). The Insomnia Severity Index, the Photosensitivity Assessment Questionnaire (PAQ), the Allodynia Symptom Checklist (ASC-12), the Hyperacusis Questionnaire, the Migraine Disability Assessment Scale (MIDAS), the COMPASS 31-autonomic symptom questionnaire, a validated post-traumatic stress disorder checklist for DSM-5 (PCL-5), a neurobehavioral and vestibular symptom Inventory (NSI), the Orthostatic Intolerance Specific Symptom Score questionnaire (OISS) for assessing orthostatic hypotension and a detailed headache symptom assessment. To assess memory, attention and mood: Trail Making (A and B), the Ray Auditory Verbal Learning Test (RAVLT), state and trait anxiety inventory (STAI, Form Y-1 and Form Y-2), the Beck Depression Inventory (BDI). Headache and TBI characteristics will be collected using case report forms containing Common Data Elements (CDE) developed by the National Institute of Neurological Disorders and Stroke (NINDS). These CDEs were developed with experts in the fields of headache and TBI in order to standardize the collection of research data to facilitate data sharing efforts and comparisons of results amongst studies. The NINDS requests that investigators use the CDEs whenever possible. For this study, the following modified CDE forms will be utilized for all subjects (excluding additional pediatric-specific elements): 1) demographics; 2) social status; 3) medical and family history; 4) behavioral history; 5) medical and family history of headache/migraine. Subjects with PTH will also complete the following CDEs: 6) headache symptoms, frequency and severity (with additional questions about worsening of symptoms with physical and cognitive exertion); 7) headache diagnosis; 8) type, place, cause and mechanism of injury; 9) neurological assessment: loss of consciousness, post-traumatic amnesia, and alteration of consciousness. In addition to the CDE forms, information will be collected on medication use for PTH and other symptoms, caffeine intake, history and symptoms of prior concussions, injury mechanism, and presence of post-concussion symptoms other than PTH. In addition, all PTH subjects will provide information on current and past use of medications used for headache/pain by indicating their usage on a list of all such medications. All PTH subjects will report their post-TBI symptoms using the 22-item Symptom Evaluation Checklist from the Sport Concussion Assessment Tool (SCAT) 5th edition.

Speech Assessment: An existing computer/mobile device based program that has been developed to assess speech amongst those with migraine and other neurologic disorders will be used to evaluate articulation, prosodic variability, phonation, and speaking rate and variation. The speech task takes 3 minutes to complete. Healthy controls will receive the same testing battery at both time-points except for those questionnaires that relate to PTH. This entire set of case report forms, questionnaires, and tests has been previously administered many times to individuals with migraine and PTH (as part of a recently completed DOD-sponsored research project) and consistently they are all completed in less than 60 minutes. Between time-point I and time-point II testing, subjects with PTH will keep a detailed daily headache diary (eDiary) documenting headache frequency, duration, intensity, headache-related functional disability, and medications used to treat headaches. Headache diaries can be completed by patients either online or in written format—as the patient prefers.

Diagnostic Imaging: T1-weighted and T2-weighted imaging will be reviewed by a board certified neuroradiologist to rule out gross structural brain abnormalities. If structural abnormalities are identified suggesting moderate or severe TBI or other brain abnormalities, these data will be excluded from further analysis.

### Post-Processing of Structural Imaging Data (T1-Weighted Imaging and DTI)

T1-weighted data: Regional measurements of gray matter thickness, volume and area will be calculated from T1-weighted imaging data using brain segmentation software (Freesurfer 5.3). The methodology for FreeSurfer is well-documented and established and includes skull stripping, automated Talairach transformation, segmentation of gray and white matter regions, intensity normalization, brain boundary tessellation, topology correction and deformation of surface structures. This automated technique outputs left and right hemisphere regional estimates of cortical thickness, volume, and area. Cortical thickness is the measured distance (in mm) between the pial surface and the white matter boundary. Regional estimates of cortical thickness (mm) volume (mm3) and area (mm2) will be calculated for cortical regions, and regional volume (mm3) will be estimated for subcortical regions. These regional measurements will be imported into SPSS 21.0 (SPSS Inc, Chicago, IL) for further calculations.

DTI data: 18 major fiber tracts will be reconstructed from DTI data using an automated technique based upon global probabilistic tractography (TRACULA). This software toolbox is freely available online and uses anatomical priors (T1-weighted data) as input. The robustness of the TRACULA software algorithm has been validated in prior papers. For each of the 18 fibertracts, tract volume, path length, axial diffusivity (AD), radial diffusivity (RD), mean diffusivity (MD), and fractional anisotropy (FA) will be calculated.

Post-Processing of Functional Imaging Data (rs-fMRI): Rs-fMRI data will be analyzed using SPM 8 (Statistical Parametric Mapping, version 8, Wellcome Department of Cognitive Neurology, Institute of Neurology, London, UK) and the toolbox DPARSF (Processing Assistant for Resting-State fMRI). SPM 8 and DPARSF are freely downloadable online and will be interfaced with MATLAB version 11.0 (Matrix Laboratory, MathWorks, Natick, Ma, USA, version 11.0). Rs-fMRI data will be pre-processed using standard procedures including the following steps: slice-time correction, motion correction, re-alignment, skull and non-brain tissue removal, spatial smoothing, and alignment to an average Montreal Neurological Institute (MNI-305) template. Further post-processing steps will include band-pass filtering, as well as removal of variance related to head motion, white matter signal and cerebrospinal fluid signal. A region of interest approach (ROI) will be used to interrogate functional connectivity patterns between each ROI and the rest of the brain. ROIs for this study have been selected based on previous findings and include 33 cortical and subcortical areas over the left and right hemisphere, which are important for pain processing or multisensory integration and have been directly implicated in mTBI or PTH. Fisher r-z transformation maps will be calculated and imported to SPSS-21.0 (Software Package for Statistical Analysis, IBM, version 21.0) for further analysis.

Arterial Spin Labeling (ASL): ASL data will be analyzed using SPM 8 and the toolbox ASLtbx. SPM 8 and ASLbtx are freely downloadable online and will be interfaced with MATLAB version 11.0. ASL perfusion data will be preprocessed using standard SPM preprocessing steps including orientation resetting, image reorienting, motion correction, and co-registration (of each subjects' T1-weighted image with the ASL images) and data smoothing. Subsequent data post-processing steps will be conducted using the ASLtbx toolbox. Statistical analyses will be conducted using a GLM design within MATLAB and SPM8.

Imaging Data Analyses for Specific Aim 1: All structural (T1-weighted, DTI) and functional (rs-fMRI, ASL) neuroimaging output metrics as well as data from clinical questionnaires will be imported to SPSS 21.0 for further analysis. Multiple regression models accounting for age, sex, depression, anxiety and other variables that significantly differ between patient cohorts will be applied to determine brain structural and functional changes in patients who transition to PPTH from patients who recover from PTH during the acute phase and from healthy control subjects.

### Expected Results

Based on published findings as well as preliminary data, there is a high likelihood that structural and functional brain features in patients at high risk for developing PPTH compared to patients that acutely recover from PTH will be determined, which is a group-based analysis and the first aim of the study. The healthy control cohort will inform the direction brain changes relative to the PTH cohort.

A highly accurate prognostic biomarker signatures to predict patients who will have persistence of PTH will be developed. The relative contributions of a) brain MRI structural and functional measures and b) clinical data, collected between 7-28 days since onset of PTH to the predictive model will be determined by two independently working machine-learning laboratories.

A biomarker signature based on neuroimaging data will be developed.

Based on previously published research, a machine-learning (ML) pipeline for structural and functional imaging data integration has been used in classifying migraineurs versus healthy controls. The general structure of this pipeline will be used and customized in this study to integrate structural and functional imaging data to predict patients at high risk for PTH persistence. The imaging features that significantly contribute to the model will be identified as potential predictors. Specifically, the pipeline will include three major steps: First, Principal Component Analysis (PCA) is applied to the features contained in each modality (e.g. T1, DTI, and rs-fMRI) for noise and dimension reduction. Second, the reduced features will be combined using a classification algorithm whose performance is optimized by a Particle Swarm Optimization (PSO) technique. Various classification algorithms can be used, such as Linear Discriminant Analysis (LDA), Quadratic Discriminant Analysis (QDA), and Support Vector Machines (SVM). The algorithm with the highest cross-validation accuracy will be used to produce the final classifier, namely the imaging-based biomarker for predicting PPTH. Third, an analyzer will identify the relative contributions of different imaging modalities and features to the prediction accuracy. This will help characterize structural and functional features of the brain that are associated with eventual PTH persistence. To avoid overfitting, 10-fold cross validation is employed.

### Expected Results

It is anticipated that the imaging classifier will have >75% cross-validation accuracy in predicting PTH persistence (similar levels of sensitivity and specificity), and <10% error bar reflecting the repeatability of the accuracy under sampling and training variability.

A biomarker signature to predict patients who will have persistence of PTH based on clinical data will be developed.

Prognostication models will be developed based on detailed clinical data from the Headache Symptom Battery and the speech samples. The relative contributions of the various clinical component data collected between 7-28 days since onset of PTH to the predictive model will be determined.

A classifier to predict PPTH from several relevant clinical variables will be developed. The approach to this problem includes 1) collecting relevant features, 2) developing clinically interpretable machine learning algorithms, 3) evaluating the algorithms using cross-validation.

### Clinical and Speech Features

Work previously done has shown the predictive value of clinical features. In addition, these features will allow for control of for potentially confounding third variables that likely correlate with symptoms (for example, anxiety and stress). Broadly speaking, the clinical measures are split into four category measures: 1) headache 2) psychological 3) cognitive, and 4) speech.

Classifier features from the first three sets of clinical measures come directly from responses to the questionnaires; however, an innovative aspect of this study is the collection of speech samples during in-clinic visits using an already-developed mobile application. The ability to share our thoughts and ideas through spoken communication is a complex and fragile process. Even the simplest verbal response requires a complex sequence of events. It requires thinking of the words that best convey the message, sequencing these words in an order that is allowed in the language, and then sending signals to the muscles required to produce speech. Even the slightest disturbance to the brain areas that orchestrate these events can manifest in speech problems. Previous work has shown that oncoming migraine attacks can be predicted up to 12 hours before onset by measuring subtle changes in speech. It is expected that there will be differences in speech patterns between individuals who go on to develop PPTH and those that do not. Furthermore, because of known complexities in speech production speech changes that track with headache, it is expected that there is predictive value in these speech parameters with respect to the task of interest. During each in-clinic research visit, study participants will use the app and provide carefully-selected speech samples from which extract six complementary feature sets that represent physical characteristics of speech will be extracted: 1) Articulation Entropy—the accuracy with which articulators (e.g. tongue, lips, palate) achieve their targets. The articulation entropy is measured using the algorithm developed by Jiao et al., 2) Rate Features—Speaking Rate, Pause Rate, Articulation Rate—the rate at which a speaker enunciates syllables in a sentence. The speaking rate is measured using an developed algorithm by Jiao et al. 2015. 3) Vowel Space Area—the area of the quadrilateral in vowel space formed by the first and second formants of the four corner vowels. Because formants, resonant peaks in the frequency spectrum of speech, relate to the kinematics of speech production (e.g. tongue position, oral cavity size/shape), vowel space area can be used to measure changes in articulatory control. The vowel space area is estimated using the algorithm in Sandoval 2013. 4) Energy Decay Slope—during a sustained phonation, measuring the rate at which a speaker's volume decreases over time. A large energy decay slope can be an indicator of fatigue. 5) Phonatory Duration—during a sustained phonation, measuring the length of time a speaker can produce a vowel sound (phonation) before stopping to take a breath. 6) Average Pitch—the fundamental frequency of a speaker's voice averaged across the duration of five sentences. After extraction of all features, a data matrix is generated with the clinical features serving as the independent variables in the classification model; the dependent variable is a binary outcome that informs whether the participant goes on to develop PPTH. This data will be used to design our classifier.

### Machine Learning Models

Clinical data is often scarce and high-dimensional. This will indeed the case here—the clinical questionnaires result in a large number of features for every participant. This results in a dataset where the sample size is smaller than the number of features. To that end, a classification schemes that simultaneously classify between the two classes and down selects the number of features to only the most relevant subset will be utilized. Two families of learning algorithms will be evaluated: elastic net regression and decision trees. Elastic net regression is a sparse extension of linear regression that penalizes models that use a large number of features or features that are correlated. Decision tree classifiers learn an optimal subset of simple rules to predict the class label. These algorithms have several benefits: 1) there is ample evidence that they can be successfully trained in cases where the feature size exceeds the sample size; 2) the resultant decision rules are easily-interpretable and 3) in the case of decision tree classifiers, clinical decisions often follow a similar paradigm. All classifiers will be implemented in Python.

### Classifier Training and Evaluation

The model will be evaluated using leave-one-out cross validation. That is, all but one sample is used to train the classifier and to set any necessary hyperparameters and evaluate the results on the remaining sample (which the model has not been trained on). This process will be repeated by iteratively replacing the held-out sample and evaluating the classifier until all data has been classified. Classification accuracy is evaluated by calculating the percent of all subjects correctly classified by the model. the development of a clinical classifier will achieve an accuracy of 75% in predicting the development of PPTH with similar levels of sensitivity and specificity is expected.

Both machine-learning laboratories will work together to fuse the biomarkers developed from neuroimaging and clinical data to develop an ensemble biomarker signature. The overall accuracy of the ensemble biomarker will be determined.

Ensemble learning is an algorithm procedure aimed at combining the predictors from multiple base classifiers into a single decision (ensemble signature). Ensemble learning will be used to combine the predictors from the imaging and clinical classifiers (considered as two base classifiers) for each patient into a final/ensemble predictor. Ensemble learning is known to outperform base classifiers used alone for situations when each base classifier is built to capture one aspect of the complete data. This is exactly our situation here. Also, ensemble learning combines prediction results from the base classifiers but not the features used to build each base classifier. This retains the integrity of the base classifiers and provides flexibility that each base classifier can be built using a classification algorithm most appropriate for the feature characteristics (e.g., imaging, clinical). A common ensemble learning approach is to combine the predictions from base classifiers by majority voting, which is equivalent to assigning equal weights to the base classifiers, i.e., each base classifier is considered to contribute equally to the final prediction. This assumption may be too strong and may need to be relaxed to be able to identify the different contributions to the final prediction from imaging and clinical data. Therefore, a weighted ensemble learning algorithm will be adopted, which computes a weighted combination of the predictions from base classifiers with weights computed in a data-driven manner using constrained quadratic programming.

It is anticipated that the combined classifier will improve the accuracy to >85% and reduce the variability to <5%.

### Validation

The dataset collected containing 100 concussed patients with acute PTH (expected ˜50 will develop PPTH) will be utilized to validate the individual and combined biomarker signatures in later parts of the study. Metrics of validation include overall accuracy in classifying patients who develop PPTH versus who recover, sensitivity (proportion of patients who develop PPTH are classified correctly), specificity (proportion of patients who have resolution of acute PTH are classified correctly), and error bars/confidence intervals of these indices. To avoid overfitting, the accuracy indices using a 10-fold cross validation will be calculated, in which the samples will be partitioned into 10 non-overlapping folds. Nine folds will be used for training a classifier, which is then used to predict on the remaining fold to which the training set is blinded. This way, none of the data that were used to train the classifier are used to test the accuracy of the classification algorithm. The cross validation will be repeated for 100 times together with bootstrap sampling to compute the error bars for the accuracy indices, which reflect the reliability of the biomarkers under sampling and training variability.

### Further Objectives

The prognostic model for PPTH that is developed during the earlier phases will be validated and optimized using a new patient cohort using an ensemble approach.

Both laboratories will validate the classification algorithm developed during the earlier parts of the study on another dataset containing 100 concussed patients with semi-acute PTH (expected ˜50 will develop PPTH). This dataset will be refer to as “B” and the previous dataset used in the study is “A”. In machine-learning (ML), a long-standing issue is that the model developed and optimized on one dataset may not work equally well on another separately collected dataset, known as the issue of reproducibility. A reproducible biomarker signature is highly desirable for generalized clinical use. Specifically, the ML classifiers developed using dataset A will be applied to the new dataset B and compute the accuracy metrics (accuracy, sensitivity, specificity) of classifying patients in B. The accuracy on B will be compared with the previously obtained accuracy on A using cross-validation. Since the accuracy on A has been obtained with error bars, the accuracy on B is considered not statistically significantly different from that on A if falling within the error bars. If not (e.g., lower than the lower bound of the accuracy on A), it indicates that there may be an issue of reproducibility of the imaging, clinical, and combined biomarker signatures on the new dataset. Subsequently, the roles of datasets A and B will be switched by using B to train the classifiers and develop the biomarkers, and then using A to test the accuracy. This procedure will identify whether lack of reproducibility exists. One common reason for lack of reproducibility is that the dataset used to train the classifier does not cover enough variability of the features in the study population so that using the classifier on a new dataset extrapolates too much leading to poor accuracy. By reversing the roles of A and B, it will enable assessment of the quality of A and B for serving as the dataset for developing the biomarkers.

### Further Refinement, Validation, and Reproducibility Assessment

There will be several possible outcomes from the previous phase of the study. There are strategies to tackle potential pitfalls for biomarker refinement: (1) In the best-case scenario, the performance of imaging, clinical, and combined biomarker signatures on the validation set is statistically equally good to that on the training set (with both A and B serving as the training set). Both datasets will be combined to train a final biomarker signature. The increased sample size will produce a more reliable biomarker signature with reduced error bars in accuracy indices. (2) If the imaging biomarker is not reproducible (and likely causing the combined biomarker signature to be non-reproducible either), first the image acquisition, quality control, and pre-processing steps performed in the two datasets will be examined and make correction if needed. If performance discrepancy still exists, it is likely because the imaging features in each dataset do not cover enough variability of the imaging features in the study population resulting in the risk of extrapolation and lack of generalizability. The two datasets will be pooled and multiple random splits of the pooled data into a training and a validation set will be performed and ML, training and validation will be re-perform. A mix of samples from the two datasets in the training set will help cover more variability of the imaging features and therefore help with the generalized performance on the validation set. (3) If the clinical biomarker is not reproducible (and likely causing the combined biomarker signature to be non-reproducible either), similar work to (2) will be performed but focusing on examining the clinical data collection instrument and speech signal analysis and feature extraction. (4) If all imaging, clinical, and combined biomarkers are not reproducible, similar work to (2) and (3) will be performed. Additionally, cases in one dataset that are wrongly classified using the trained classifier on another dataset will be identified. They will be examined in terms of data collection and feature discrepancy compared with the correctly classified cases and the training cases to either make correction or identify outliers. Regardless of the potential scenarios, this study will generate 1) improved, standardized data collection, quality control, and pre-processing steps for imaging and clinical data; 2) a robust biomarker signature optimized for better generalizable performance.

### Findings

Currently, there are no adequate methods to predict whether a patient with PTH will have resolution of headaches during the acute phase or will have persistence of PTH. The primary goal of this study is to use clinical and imaging data collected in the semi-acute post-concussion setting to predict the development of PPTH. The goal of this study is to identify neuropathologic prognostic biomarkers for the development of PPTH using clinical data and structural and functional neuroimaging and assess the predictive value of neuroimaging combined with clinical data for predicting which patients are at high risk for PPTH. The ability to predict who will develop PPTH would allow clinicians to determine how aggressive to be with early treatment and thus could directly impact patient care and outcomes.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

### Computing System

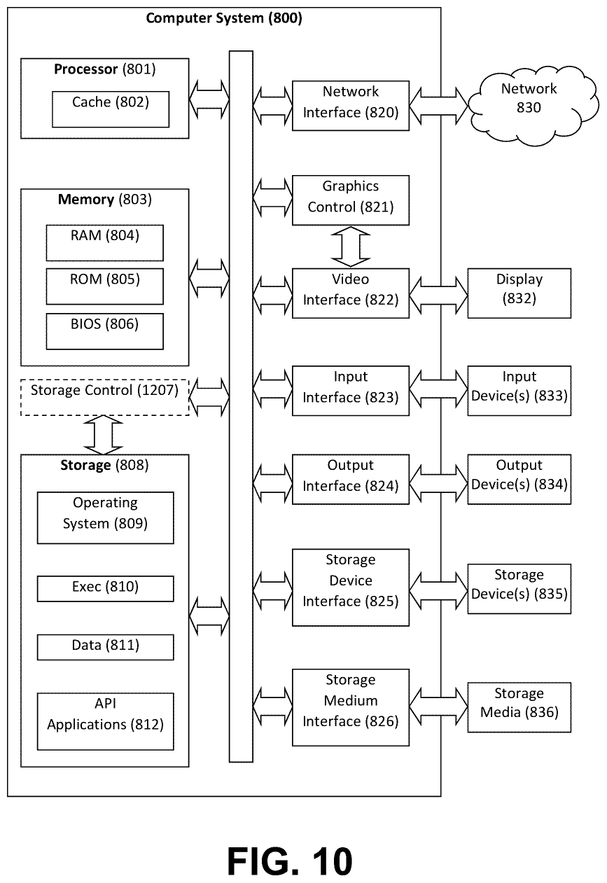

Referring to FIG. 10, a block diagram is shown depicting an exemplary machine that includes a computer system 1200 (e.g., a processing or computing system) within which a set of instructions can execute for causing a device to perform or execute any one or more of the aspects and/or methodologies for static code scheduling of the present disclosure. The components in FIG. 10 are examples only and do not limit the scope of use or functionality of any hardware, software, embedded logic component, or a combination of two or more such components implementing particular embodiments.

Computer system 1200 may include one or more processors 1201, a memory 1203, and a storage 1208 that communicate with each other, and with other components, via a bus 1240. The bus 1240 may also link a display 1232, one or more input devices 1233 (which may, for example, include a keypad, a keyboard, a mouse, a stylus, etc.), one or more output devices 1234, one or more storage devices 1235, and various tangible storage media 1236. All of these elements may interface directly or via one or more interfaces or adaptors to the bus 1240. For instance, the various tangible storage media 1236 can interface with the bus 1240 via storage medium interface 1226. Computer system 1200 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 1200 includes one or more processor(s) 1201 (e.g., central processing units (CPUs) or general purpose graphics processing units (GPGPUs)) that carry out functions. Processor(s) 1201 optionally contains a cache memory unit 1202 for temporary local storage of instructions, data, or computer addresses. Processor(s) 1201 are configured to assist in execution of computer readable instructions. Computer system 1200 may provide functionality for the components depicted in FIG. 10 as a result of the processor(s) 1201 executing non-transitory, processor-executable instructions embodied in one or more tangible computer-readable storage media, such as memory 1203, storage 1208, storage devices 1235, and/or storage medium 1236. The computer-readable media may store software that implements particular embodiments, and processor(s) 1201 may execute the software. Memory 1203 may read the software from one or more other computer-readable media (such as mass storage device(s) 1235, 1236) or from one or more other sources through a suitable interface, such as network interface 1220. The software may cause processor(s) 1201 to carry out one or more processes or one or more steps of one or more processes described or illustrated herein. Carrying out such processes or steps may include defining data structures stored in memory 1203 and modifying the data structures as directed by the software.

The memory 1203 may include various components (e.g., machine readable media) including, but not limited to, a random access memory component (e.g., RAM 1204) (e.g., static RAM (SRAM), dynamic RAM (DRAM), ferroelectric random access memory (FRAM), phase-change random access memory (PRAM), etc.), a read-only memory component (e.g., ROM 1205), and any combinations thereof. ROM 1205 may act to communicate data and instructions unidirectionally to processor(s) 1201, and RAM 1204 may act to communicate data and instructions bidirectionally with processor(s) 1201. ROM 1205 and RAM 1204 may include any suitable tangible computer-readable media described below. In one example, a basic input/output system 1206 (BIOS), including basic routines that help to transfer information between elements within computer system 1200, such as during start-up, may be stored in the memory 1203.

Fixed storage 1208 is connected bidirectionally to processor(s) 1201, optionally through storage control unit 1207. Fixed storage 1208 provides additional data storage capacity and may also include any suitable tangible computer-readable media described herein. Storage 1208 may be used to store operating system 1209, executable(s) 1210, data 1211, applications 1212 (application programs), and the like. Storage 1208 can also include an optical disk drive, a solid-state memory device (e.g., flash-based systems), or a combination of any of the above. Information in storage 1208 may, in appropriate cases, be incorporated as virtual memory in memory 1203.

In one example, storage device(s) 1235 may be removably interfaced with computer system 1200 (e.g., via an external port connector (not shown)) via a storage device interface 1225. Particularly, storage device(s) 1235 and an associated machine-readable medium may provide non-volatile and/or volatile storage of machine-readable instructions, data structures, program modules, and/or other data for the computer system 1200. In one example, software may reside, completely or partially, within a machine-readable medium on storage device(s) 1235. In another example, software may reside, completely or partially, within processor(s) 1201.

Bus 1240 connects a wide variety of subsystems. Herein, reference to a bus may encompass one or more digital signal lines serving a common function, where appropriate. Bus 1240 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1200 may also include an input device 1233. In one example, a user of computer system 1200 may enter commands and/or other information into computer system 1200 via input device(s) 1233. Examples of an input device(s) 1233 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 1233 may be interfaced to bus 1240 via any of a variety of input interfaces 1223 (e.g., input interface 1223) including, but not limited to, serial, parallel, game port, USB, FIREWIRE, THUNDERBOLT, or any combination of the above.

In particular embodiments, when computer system 1200 is connected to network 1230, computer system 1200 may communicate with other devices, specifically mobile devices and enterprise systems, distributed computing systems, cloud storage systems, cloud computing systems, and the like, connected to network 1230. Communications to and from computer system 1200 may be sent through network interface 1220. For example, network interface 1220 may receive incoming communications (such as requests or responses from other devices) in the form of one or more packets (such as Internet Protocol (IP) packets) from network 1230, and computer system 1200 may store the incoming communications in memory 1203 for processing. Computer system 1200 may similarly store outgoing communications (such as requests or responses to other devices) in the form of one or more packets in memory 1203 and communicated to network 1230 from network interface 1220. Processor(s) 1201 may access these communication packets stored in memory 1203 for processing.

Examples of the network interface 1220 include, but are not limited to, a network interface card, a modem, and any combination thereof. Examples of a network 1230 or network segment 1230 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1230, may employ a wired and/or a wireless mode of communication. In general, any network topology may be used.

Information and data can be displayed through a display 1232. Examples of a display 1232 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 1232 can interface to the processor(s) 1201, memory 1203, and fixed storage 1208, as well as other devices, such as input device(s) 1233, via the bus 1240. The display 1232 is linked to the bus 1240 via a video interface 1222, and transport of data between the display 1232 and the bus 1240 can be controlled via the graphics control 1221. In some embodiments, the display is a video projector. In some embodiments, the display is a head-mounted display (TIMID) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 1232, computer system 1200 may include one or more other peripheral output devices 1234 including, but not limited to, an audio speaker, a printer, a storage device, and any combinations thereof. Such peripheral output devices may be connected to the bus 1240 via an output interface 1224. Examples of an output interface 1224 include, but are not limited to, a serial port, a parallel connection, a USB port, a FIREWIRE port, a THUNDERBOLT port, and any combinations thereof.

In addition or as an alternative, computer system 1200 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3@, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

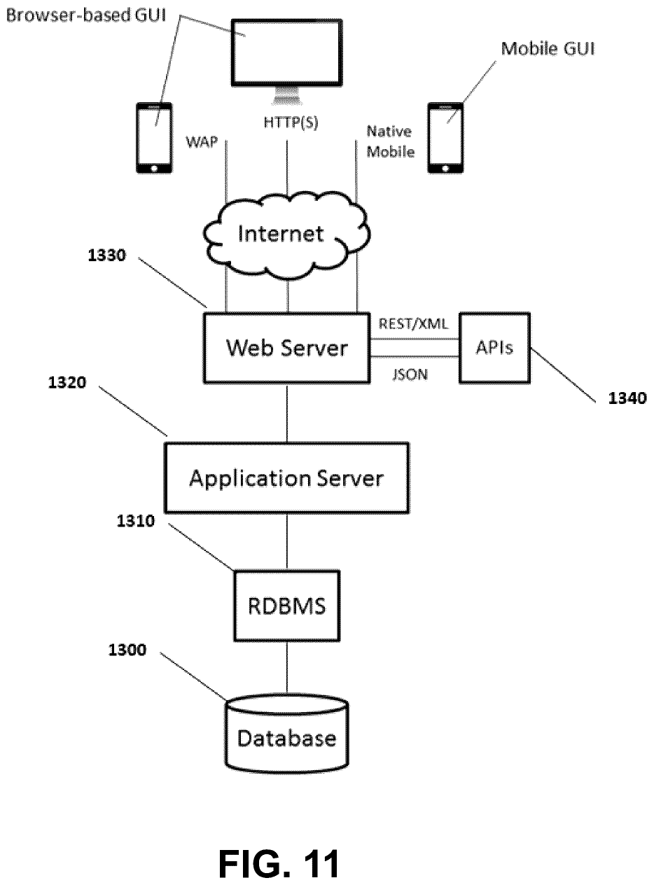

Referring to FIG. 11, in a particular embodiment, an application provision system comprises one or more databases 1300 accessed by a relational database management system (RDBMS) 1310. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 1320 (such as Java servers, NET servers, PUP servers, and the like) and one or more web servers 1330 (such as Apache, IIS, GWS and the like). The web server(s) optionally expose one or more web services via app application programming interfaces (APIs) 1340. Via a network, such as the Internet, the system provides browser-based and/or mobile native user interfaces.

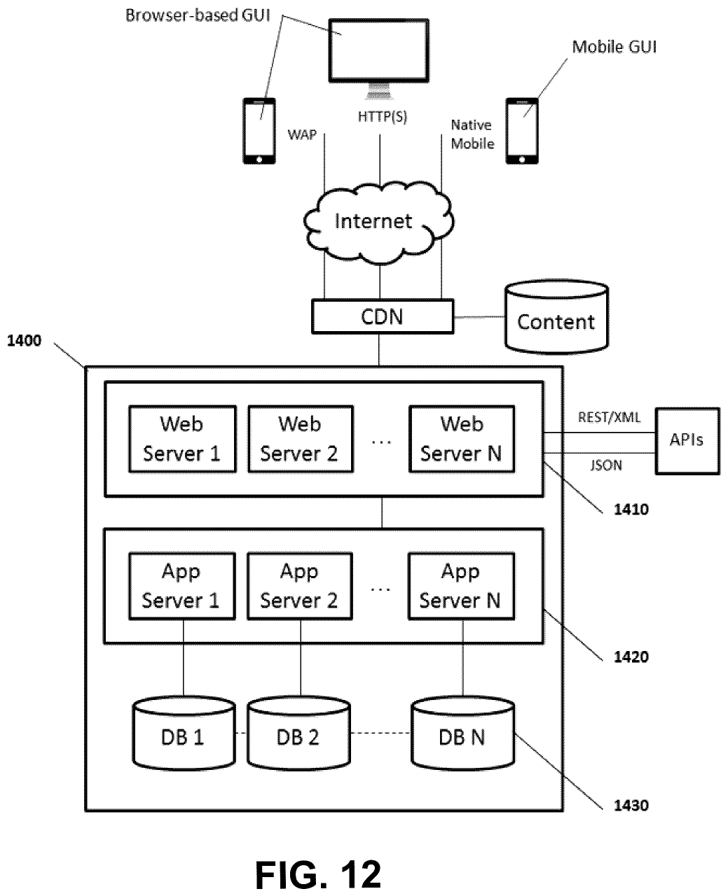

Referring to FIG. 12, in a particular embodiment, an application provision system alternatively has a distributed, cloud-based architecture 1400 and comprises elastically load balanced, auto-scaling web server resources 1410 and application server resources 1420 as well synchronously replicated databases 1430.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of DEL information and associated experimental data collected for one or more conditions. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A method for evaluating a subject for brain injury, comprising:
(a) receiving input data comprising one or more acoustic features and/or one or more linguistic features extracted from audio data obtained for said subject;
(b) processing the input data using a machine learning module configured to evaluate acoustic and/or linguistic features;
(c) generate an evaluation of said subject based on said processing of said input data using said machine learning module, said evaluation comprising an indication of brain injury.

2. The method of claim 1, further comprising pre-determining said subject has suffered an injury affecting brain health and selecting said subject for said evaluation.

3. The method of claim 2, wherein said injury comprises traumatic brain injury or mild traumatic brain injury.

4. The method of claim 1, further comprising prompting said subject for said audio data.

5. The method of claim 4, wherein said subject is shown one or more sentences and prompted to read said one or more sentences.

6. The method of claim 5, further comprising capturing said audio data using a microphone, said audio data comprising an audio recording of said subject reading said one or more sentences.

7. The method of claim 6, further comprising processing said audio data to extract said one or more acoustic features and/or one or more linguistic features.

8. The method of claim 1, wherein said one or more acoustic features comprises one or more of sentence speaking rate, average pitch value, pitch variance, vowel and/or consonant articulation precision, vowel space area, or spontaneous pause rate.

9. The method of claim 1, wherein said one or more linguistic features comprises one or more of LIWC feature, part of speech feature, language complexity feature, grammatical constituent feature, or phrase formation type.

10. The method of claim 9, wherein said LIWC feature comprises a calculation of words categorized according to one or more of attention and/or focus, emotion, social relationship, thinking style, or cognitive complexity.

11. The method of claim 9, wherein said part of speech feature comprises a percentile of total words for a part of speech.

12. The method of claim 9, wherein said language complexity feature comprises a Yngve Depth, Brunet Index, or Honore Statistic.

13. The method of claim 9, wherein said part of speech feature comprises an appearance time of a noun phrase, an appearance time of a verb phrase, or an appearance time of a noun phrase that contains only one noun.

14. The method of claim 1, wherein said machine learning module comprises a model trained using a training data set comprising acoustic and/or linguistic features for a plurality of individuals, wherein each of said plurality of individuals is labeled according to said indication of brain injury.

15. The method of claim 14, wherein said indication of brain injury is associated with traumatic brain injury.

16. The method of claim 14, wherein said indication of brain injury comprises one or more symptoms of traumatic brain injury.

17. The method of claim 14, wherein said indication of brain injury is associated with a severity of traumatic brain injury.

18. The method of claim 14, wherein said indication of brain injury comprises a prediction of a future symptom of traumatic brain injury.

19. The method of claim 1, wherein said indication of brain injury differentiates between whether said subject will recover from an acute traumatic brain injury or whether said subject will suffer one or more symptoms of said acute traumatic brain injury.

20. The method of claim 19, wherein said one or more symptoms of acute traumatic brain injury comprises post-traumatic headache or persistent post-traumatic headache.
